(19) United States
(12) Patent Application Publication
(10) Pub. No.: US 2002/0052354 A1
(43) Pub. Date: May 2, 2002

(54) Paramagnetic DOTA derivatives, pharmaceutical agents that contain the latter, process for their production, and their use for MR imaging of necrosis and infarction

(75) Inventors: Johannes Platzek, Berlin (DE); Heribert Schmitt-Willich, Berlin (DE); Ulrich Niedballa, Berlin (DE); Hanns-Joachim Weinmann, Berlin (DE); Wolfgang Ebert, Mahlow (DE)

Correspondence Address: MILLEN, WHITE, ZELANO & BRANIGAN, P.C., Arlington Courthouse Plaza I, Suite 1400, 2200 Clarendon Boulevard, Arlington, VA, 22201, US

(73) Assignee: Schering AG

(21) Appl. No.: 09/759,364

(22) Filed: Jan. 16, 2001

Related U.S. Application Data
(60) Provisional application No. 60/178,316, filed on Jan. 27, 2000.

(51) Int. Cl. A61K 31/555; C07F 19/00

(52) U.S. Cl. 514/185; 534/15; 540/465

(57) ABSTRACT

The invention relates to compounds of general formula I

Ar-(L-K)ₙ

in which

K means a cyclic non-radioactive metal complex of the DOTA-type,

L means a linker,

Ar means an aromatic radical, which contains a polycondensated aromatic hydrocarbon, and

n means the numbers 1 or 2,

diagnostic agents that contain these compounds, their use for MR imaging of necrosis and infarction as well as process for the production of these compounds and agents.

Benefit is claimed of the filing date of Jan. 27, 2000 of Provisional application No. 60/178,316, whose entire disclosure is incorporated by reference herein.

The invention relates to the subject that is characterized in the claims, i.e., paramagnetic DOTA derivatives, pharmaceutical agents that contain the latter, process for their production, and their use for MR imaging of necrosis and infarction.

Detecting, locating, and monitoring necroses or infarctions is an important area in medicine. Thus, myocardial infarction is not a stationary process, but rather a dynamic process, which extends over a prolonged period—weeks to months. Infarction runs in phases, which are not strictly separate from one another but rather overlap. The first phase, the development of myocardial infarction, comprises the 24 hours after the infarction, during which the destruction spreads like a wave from the subendocardium to the myocardium. The second phase, the already existing infarction, comprises the stabilization of the area in which fiber formation (fibrosis) takes place as a healing process. The third phase, the healed infarction, begins after all destroyed tissue is replaced by fibrous scar tissue. During this period, extensive restructuring takes place.

Up until now, no specific and reliable process has been known that makes it possible to determine the current phase of a myocardial infarction in a live patient. To evaluate a myocardial infarction, it is of decisive importance to know how large the proportion of the tissue is that is extinct (lost) in the infarction and at what point the loss has taken place since the type of treatment depends on this knowledge. Infarctions take place not only in the myocardium but also in other tissues, especially in the brain.

While the infarction can be healed to a certain extent, in the case of a necrosis, locally limited tissue death, only the harmful sequelae for the residual organism can be prevented or at least mitigated. Necroses can occur in many ways: from injuries, chemicals, oxygen deficiency, or radiation.

As in the case of infarction, knowing the extent and type of necrosis is important for subsequent medical treatment. Attempts have thus already been made to improve the detection and locating of infarctions and necroses by using contrast media in the case of noninvasive processes such as scintigraphy or MRI. In literature, attempts to use porphyrins for necrosis imaging take up a good deal of space. The results that have been achieved, however, paint a contradictory picture.

Winkelman and Hayes in Nature, 200, 903 (1967) describe the fact that Mn-5,10,15,20-tetrakis (4-sulfonatophenyl)-porphyrin (TPPS) accumulates selectively in the necrotic portion of a tumor. Lyon et al., Magn. Res. Med. 4, 24 (1987), however, observed that Mn-TPPS is distributed in the body, specifically in the kidney, liver, tumor and only in a small proportion in the muscles. In this case, it is advantageous for the concentration in the tumor to reach its maximum only on the fourth day, and this occurred only after the authors had increased the dose to 0.2 mmol/kg. The authors therefore also speak of a clearly nonspecific uptake of TPPS into the tumor.

Bockhorst et al. again report in Acta Neurochir. 1994 [Suppi.] 60, 347 that MnTPPS selectively binds to tumor cells. Foster et al., J. Nucl. Med. 26 756 (1985) in turn found that In-111 5,10,15,20-tetrakis (4-N-methyl-pyridinium)-porphyrin (TMPyP) does not accumulate in the necrotic part, but rather in the living edge layers.

This does not necessarily indicate that a porphyrin-type-tissue-dependent interaction exists.

In Circulation, Vol. 90, No. 4, 1994, Part 2, Page 1468, Abstr. No. 2512, Ni et al. report that they can readily visualize infarction areas with an Mn-tetraphenyl-porphyrin (Mn-TTP) and a Gd-mesoporphyrin (in DE 4232925 Example Ic) (Gd-MP).

Both substances are the subject of Application WO 95/31219.

In DE 19824653, radiolabeled mesoporphyrin-IX derivatives are described as necrosisaffine compounds and are used for radiation therapy.

In the case of scintigraphic processes, the dose that is used for diagnostic purposes is in the nanomol range. The compatibility of the substances therefore plays only a subordinate role. With MR imaging, however, the dose is in the millimol range. Here, compatibility plays a quite decisive role.

The small acute compatibilities (LD50) that are determined for MnTPP or MnTPPS rule out their use in humans.

In addition, porphyrins—such as, e.g., Gd-mesoporphyrin—tend to be deposited in the skin, which results in photosensitization and discoloration. These effects can last for days or even weeks. In the case of scintigraphic processes, this effect would be unimportant because of the low dose. Broad use of scintigraphic processes, however, is contraindicated owing to the fact that the resolution of a gamma camera is very much lower than that which can be achieved with MR imaging.

For MR imaging of myocardial infarction, the Gd-complexes of DTPA were also used (K. Bockhorst et al., Acta Neurochir. (1997) Suppl., 60:347-349); De Roos et al., Radiology 1989; 172:717-720) and its bis(methylamide) (M. Saeed et al., Radiology, 1992; 182:675-683). It turned out that both contrast media make it possible to differentiate between healthy and infarcted tissue only in a narrow time window. Comparable results were also obtained with the manganese compound of DTPA (Immunomedics, WO 94/22490) and DPDP (Radiology 1989; 172:59-64).

Weissleder et al., Radiology 1992; 182:675-683, who coupled antimyosin to iron oxides (MION), achieved a considerable improvement. Owing to its specific structure, this contrast medium is not suitable for necrosis imaging.

There is therefore an urgent need to have compounds for MR imaging of infarction and necrosis that:

are very well-tolerated,

are not phototoxic,

are chemically stable,

are completely excreted,

accumulate in necroses,

are not concentrated in the skin,

have a high relaxivity,

exhibit high water solubility,

provide a wide time window for measurement,

make possible good differentiation between healthy and necrotic/infarcted tissue.

The object of the invention is achieved, surprisingly enough, by the compounds of general formula I

Ar-(L-K)ₙ (I)

in which

K: means a cyclic non-radioactive metal complex of the DOTA-type,

L: means a linker,

Ar means an aromatic radical, which contains a polycondensated aromatic hydrocarbon,

n: means the numbers 1 or 2.

Polycondensated aromatic hydrocarbons are, as it is commonly know, compounds like e.g. naphthalene, fluorene, anthracene, phenanthrene. Preferably, the compounds of general formula (J) contain one or two polycondensated aromatic hydrocarbons. In addition, they can contain more phenyl groups.

Ar preferably stands for a radical

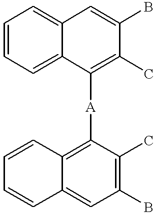

with the meaning

A: a direct bond,

a methylene group —CH₂—,

a dimethylene ether group —CH₂—O—CH₂—,

B: a hydrogen atom,

a carbonyl group —CO—,

C: a hydroxyl group —OH,

an oxygen group —O—.

an ether group —OR¹, in which R¹ means an alkyl radical with 1-3 carbon atoms, whereby substituents B and C in the molecule are respectively identical,

for a radical

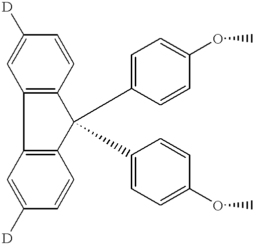

with the meaning

D: a hydrogen atom,

an ether group —OR¹, with R¹ in the above-mentioned meaning,

for a radical

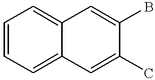

with the meaning

B and C as described above,

for a radical

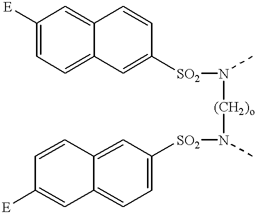

with the meaning

E: a hydrogen atom,

an ether group —OR¹,

a dialkylamino group N(¹)₂, whereby R¹ has the above-mentioned meaning,

o: a number between 2-10,

for a radical

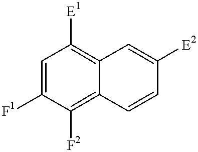

with the meaning

E¹, E²: independently of one another, in the meaning of E,

F¹, F²: independently of one another, for a hydrogen atom H or the radicals

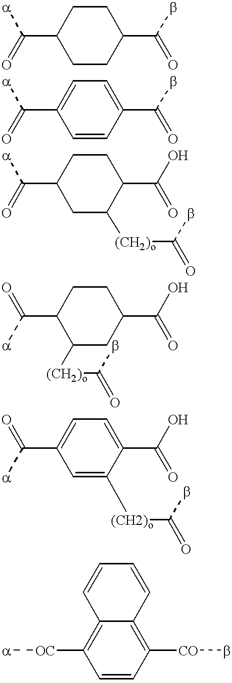

with o in the above-mentioned meaning,

and provided that one of substituents F¹ or F² stands for a hydrogen atom and that a refers to the binding site orientated to the aromatic compounds and β refers to the binding site orientated to the metal complex.

K: preferably stands for a metal complex of general formula II

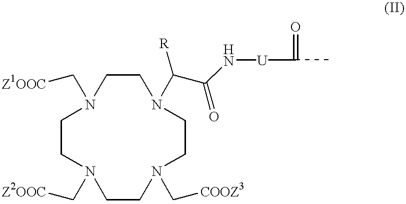

with the meaning

R: a hydrogen atom,

a methyl group,

Z¹, Z², Z³: a metal ion equivalent of the atomic numbers 25, 26 as well as 58-70,

U: a C₁-C₁₀ carbon chain, linear or branched, saturated or unsaturated, optionally interrupted by 1-2 oxygen atoms, by a phenylene group, by a cyclohexylidene group, by one or two groups —NH—CO— or —CONH—, optionally substituted with one to two —CO₂H groups, with one to three hydroxyl groups, one to three methoxy or alkoxy groups,

or for a metal complex of general formula III

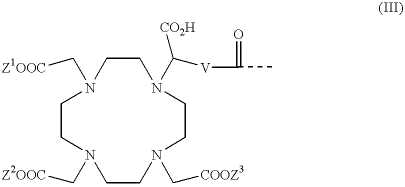

with the meaning

Z¹, Z², Z³: as indicated above.

V: a phenylene, phenylenoxymethyl- -δ-C₆H₄—O—CH₂-γ group whereby γ indicates the binding site orientated to the aromatic compound and 8 indicates the binding site orientated to the metal complex,

a C₁-C₂₀ carbon chain, linear or branched, saturated or unsaturated, optionally interrupted by one to two oxygen atoms, by a phenylene group, by a cyclohexylidene group, by one or two groups —NH—CO— or CONH—, optionally substituted with one to two —CO₂H groups with one to three hydroxyl groups, one to three methoxy or alkoxy groups.

L preferably stands for a linker in the meaning of a hydrazine group —NHNH, a C₂-C₂₀ carbon chain with terminal —NH, which can be linear or branched, saturated or unsaturated and optionally is interrupted by 1-6 oxygen atoms, 1-2 phenylene groups, 1-2 cyclohexylidene groups, 1-2 groups —NH—CO— or —CONH—, 1-2 groups —CH₂CONHNH— or —NHNHCOCH₂— and optionally is substituted with 1-2 hydroxyl groups, with 1-2 methoxy groups, with 1-2 carboxy groups.

Acid groups that are optionally present in the molecule optionally are present as salts of organic and/or inorganic bases or amino acids or amino acid amides.

Preferred radicals for L are:

—NH—NH—

γ-CH₂—CONH—NH-δ

—NH—CH₂CH₂—NH—

—NH CH₂CH₂CH₂CH₂—NH—

—NH—(CH₂)₃—NH—

—NH—(CH₂)₅—NH—

—NH—(CH₂)₂—O—(CH₂)₂—NH—

γ-NH—(CH₂)k-CONH—(CH₂)m-NH-δmit k=1-10; m=0-10,

γ-NH—(CH₂CH₂O)₂×CH₂CH₂NH-δ

[Key: mit=with]

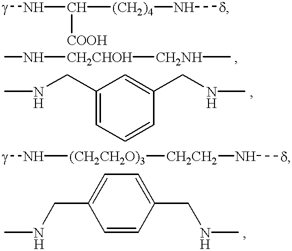

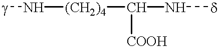

U preferably stands for the group:

—CH₂—

—CH₂CH₂—

—C₆H₄—

—CH₂—O—CH₂CH₂—

Especially preferred in this case is the —CH₂— group.

V preferably stands for a group:

—CH₂—O—C₆H₄—

—C₆H₄—

—CH₂CH₂—

—CH₂—

The compounds according to the invention meet the requirements (see above) that are imposed on diagnostic agents for imaging of necrosis and infarction.

Surprisingly enough, the complexes according to the invention show a considerably higher relaxivity compared to the previously known, structurally similar compounds. Since the relaxivity can be considered as a yardstick for the contrast medium action of a compound, a comparable, positive signal effect even at a low dose can be carried out with use of the complexes according to the invention in the area of NMR diagnosis. This significantly increases the safety margin, for which the product of relaxivity and compatibility can be considered as a guide value.

The carboxyl groups, which are not required for the complexing of metal ions, optionally can be present as esters, as amides or as salts of inorganic or organic bases. Suitable ester radicals are those with 1 to 6 C atoms, preferably the ethyl esters. Suitable inorganic cations are, for example, the lithium ion and the potassium ion and especially the sodium ion. Suitable cations of organic bases are those of primary, secondary or tertiary amines, such as, for example, ethanolamine, diethanolamine, morpholine, glucamine, N,N-dimethylglucamine, especially meglumine.

For use of the compounds of general formula I in NMk diagnosis, the central ion of complex K must be paramagnetic. These are especially the divalent and trivalent ions of the elements of atomic numbers 25, 26 and 58-70.

Preferred are iron, manganese, and gadolinium.

Especially preferred is gadolinium.

The production of the compounds of general formula I according to the invention

Ar(L-K) (I)

is carried out according to the methods that are known to one skilled in the art by reaction of compounds of general formula IV

Ar(L-H)ₙ (IV)

with complexes or complexing agents of general formula V

K-X′ (V)

which

Ar, L, K and n have the already described meaning, and

X′ stands for a hydroxyl group or a group that activates the carboxylic acid, such as, e.g.,

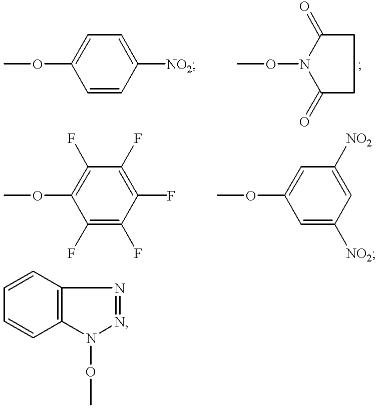

The reaction is carried out according to the methods of amide formation of active ester and amine that are known to one skilled in the art, as is described in, for example, WO 98/24775. For this purpose, metal carboxylic acid amides are obtained from acid and amine,. optionally without isolation of the active ester.

In this case, a mixture of metal complex carboxylic acid and at least one solubilizing substance in dimethyl sulfoxide is pretreated with a dehydrating reagent, optionally with the addition of a coupling adjuvant, and then reacted with an amine.

The reaction of IV and V to I can also be carried out so that the complexing agents are present in protected form, the coupling to I is performed, and then, after the protective groups are cleaved, the metal is introduced.

The introduction of the desired metal ions is carried out as was disclosed in, e.g., Patents EP 71564, EP 130934 and DE-3401052, by the metal oxide or a metal salt (for example, the nitrate, acetate, carbonate, chloride or sulfate) of the element of the desired atomic numbers being dissolved or suspended in water and/or a lower alcohol (such as methanol, ethanol or isopropanol) and reacted with a solution or suspension of the equivalent amount of the complexing agent of general formula II or III (with Z¹-Z³ in the meaning of hydrogen atoms).

The cleavage of the protective groups is carried out according to the processes that are known to one skilled in the art, for example by hydrolysis, hydrogenolysis, alkaline saponification of esters with alkali in aqueous-alcoholic solution at temperatures of 0° C. to 50° C., acid saponification with mineral acids or in the case of, e.g., tert-butyl esters with the aid of trifluoroacetic acid. [Protective Groups in Organic Synthesis, 2nd Edition, T. W. Greene and P. G. M. Wuts, John Wiley and Sons, Inc. New York, 1991].

For the production of aromatic amines of general formula IV, the procedure starts from carboxylic acids or hydroxy compounds that contain aromatic compounds like e.g. the ones which are available commercially:

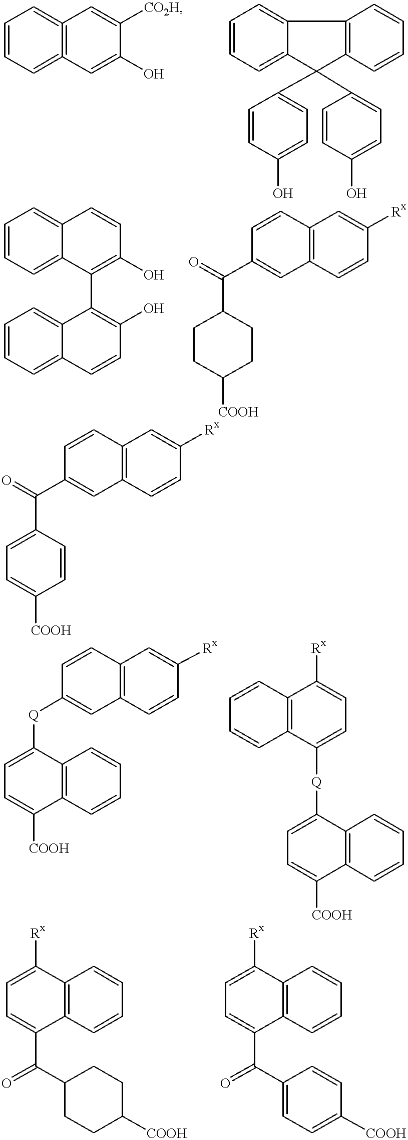

R^{x}=—OCH₃, —N(CH₃)₂

Q=NH, O, CO, SO, SO₂, S

If the carboxylic acids that contain aromatic compounds are not commercially available, they are produced by the processes that are known by one skilled in the art, e.g., by acylation of naphthalenes by corresponding acyl chlorides in the presence of Lewis acids (e.g., Pivsa-Art et al., J. Chem. Soc. Perkin Trans. 1, 1703-1707 (1994), or by Grignard reactions of the corresponding commercially available bromonaphthalenes (e.g., Kharasch, M. S. and Reinmuth, O. Grignard Reactions of Nonmetallic Substances Constable and Company, Ltd. Prentice-Hall Inc., 1954).

For the introduction of radical R^{x} in the meaning of dialkylamino, see, for example, Hoeve, W. et al., J. Org. Chem. 58, 5101-5106 (1993).

For the reaction of carboxylic acid that contains aromatic compounds to the educts of general formula IV, reference can be made to the methods that are known to one skilled in the art [e.g., Houben-Weyl, Methoden der Organischen Chemie [Methods of Organic Chemistry], Georg Thieme Verlag, Stuttgart, Volume 15/2 (1974)].

As diamines, there can be mentioned by way of example:

H₂NNH₂

H₂NCH₂CH₂NH₂,

H₂N—CH—(CH₂)₄—NH₂

COOH

In the case of hydroxy compounds, the latter can be converted into the corresponding ether carboxylic acids according to methods that are known in the literature, e.g., (Houben-Weyl, Volume VI/3, Part A, Georg Thieme Verlag, Stuttgart, 1965) by alkylation with, e.g., halocarboxylic acids, and the ether carboxylic acids are then reacted to the desired educts of Formula IV as described above.

The synthesis of the starting compounds of general formula V is described in WO 98/24774.

The production of the pharmaceutical agents according to the invention is also carried out in a way that is known in the art by the complex compounds according to the invention—optionally with the addition of the additives that are commonly used in galenicals—being suspended or dissolved in aqueous medium and then the suspension or solution optionally being sterilized. Suitable additives are, for example, physiologically harmless buffers (such as, e.g., tromethamine), small additions of complexing agents (such as, e.g., diethylenetriaminepentaacetic acid) or, if necessary, electrolytes such as, e.g., sodium chloride, or, if necessary, antioxidants, such as, e.g., ascorbic acid.

If suspensions or solutions of the agents according to the invention in water or in physiological salt solution are desired for enteral administration or for other purposes, they are mixed with one or more adjuvant(s) that are commonly used in galenicals (e.g., methylcellulose, lactose, mannitol) and/or surfactant(s) (e.g., lecithins, Tween®, Myrj®) and/or flavoring substances for taste correction (e.g., ethereal oils).

Basically, it is also possible to produce the pharmaceutical agents according to the invention even without isolating the complex salts. In any case, special care must be taken to perform the chelation in such a way that the salts according to the invention and salt solutions are virtually free of noncomplexed metal ions that have a toxic action.

This can be ensured, for example, with the aid of color indicators, such as xylenol orange, by control titrations during the production process. The invention therefore also relates to a process for the production of complex compounds and their salts. As a final precaution, there remains purification of the isolated complex salt.

The pharmaceutical agents according to the invention contain preferably 20 μmol/L to 200 mmol/L of the complex salt and are generally dosed in amounts of 1 μmol to 2 mmol/kg of body weight, both in their use for M imaging of necrosis and infarction and for therapy monitoring using MRI diagnosis. They are intended for enteral and parenteral administration or are administered with the methods of interventional radiology.

The agents according to the invention meet the varied requirements for suitability as agents for MRI contrast media. After administration, they are thus extremely well suited for enhancing the informational value of the image that is obtained with the aid of a nuclear spin tomograph by increasing the signal intensity. They also show the high effectiveness that is necessary to load the body with the smallest possible amounts of foreign substances and the good compatibility that is necessary to maintain the noninvasive nature of the studies.

The compounds of general formula I are also suitable for visualizing the intravascular space (blood-pool).

The good water-solubility of the agents according to the invention allows the production of highly concentrated solutions to ensure that the volume burden of the circulation is kept within justifiable limits and to offset the dilution by bodily fluids. In addition, the agents according to the invention have not only high stability in vitro but also surprisingly high stability in vivo, so that a release or an exchange of the ions, which are inherently toxic and not covalently bonded in the complexes, can be disregarded within the time that it takes for the contrast media to be completely excreted.

The invention is explained by the examples below.

### EXAMPLE 1

N-[1,4,7-Tris(carboxylatomethyl)-1,4,7,10-tetraazacyclododecane-Gd complex-10-(3-aza-4-oxo-hexan-5-ylic)-acid]-N′-[(3-hydroxy)-2-napthoic acid]dihydrazide

20 g (31.76 mmol) of the Gd complex of 10-(4-carboxy-1-methyl-2-oxo-3-aza-butyl)-1,4,7,10-tetraaza-cyclododecane-1,4,7-triacetic acid (Gd-Gly-Me-DOTA, DE 19652386), 2.69 g (63.52 mmol) of lithium chloride and 3.66 g (31.76 mmol) of N-hydroxysuccinimide are dissolved in 200 ml of dimethyl sulfoxide while being heated slightly. Then, 6.42 g (31.76 mmol) of 2-hydroxy-3-napthoic acid hydrazide is added, and it is stirred for 20 minutes at room temperature. It is cooled to 10IC, and 9.83 g (47.64 mmol) of N,N′-dicyclohexylcarbodiimide is added, and it is stirred overnight at room temperature. The reaction solution is poured into a mixture that consists of 1000 ml of acetone/1000 ml of diethyl ether, and the precipitated solid is filtered off. Further purification is carried out by RP-18 chromatography (mobile solvent: gradient that consists of water/acetonitrile/tetrahydrofuran). After the fractions are concentrated by evaporation, 21.46 g (83% of theory) of a colorless, amorphous solid is obtained.

Water content: 7.8%

Elementary analysis (relative to anhydrous substance):

Cld: C 44.27 H 4.71 Gd 19.32 N 12.05

Fnd: C 44.41 H 4.82 Gd 19.20 N 11.96

### EXAMPLE 2

N-[1,4,7-Tris(carboxylatomethyl)-1,4,7,10-tetraazacyclododecane-Gd-complex-10-(3-aza-4-oxo-hexan-5-ylic)-acid]-N′-[(3-methoxy)-2-napthoic acid]dihydrazide

2.12 g (20 mmol) of sodium carbonate is added to 5 g (6.14 mmol) of the title compound of Example 1, dissolved in 30 ml of water. 1.01 g (8 mmol) of dimethyl sulfate is added in drops at 0° C., and then it is stirred for 24 hours at room temperature. It is set at pH 7.4 with concentrated hydrochloric acid and evaporated to the dry state in a vacuum. The residue is purified on RP-18 (mobile solvent: gradient that consists of water/ acetonitrile/tetrahydrofuran).

Yield: 4.98 g (98% of theory) of an amorphous solid.

Water content: 9.1%

Elementary analysis (relative to anhydrous substance):

Cld: C 44.97 H 4.87 Gd 18.99 N 11.84

Fnd: C 45.10 H 5.00 Gd 19.18 N 11.97

### EXAMPLE 3

N,N-Bis[1,4,7-tris(carboxylatomethyl)-1,4,7,10-tetraazacyclododecane-Gd-complex-10-(3-aza-4-oxo-hexan-5-ylic)-acid]-4,4′-methylene-N′,N′-bis[(3-hydroxy)-2-napthoic acid]-tetrahydrazide

10 g (12.29 mmol) of the title compound of Example 1 is refluxed together with 50 ml of acetic acid/50 ml of water, 20 g (243.8 g mmol) of sodium acetate and 0.37 g (12.3 mmol) of formaldehyde (as 30% aqueous solution) for 3 hours. It is allowed to cool to room temperature, set at pH 7.5 with 10% aqueous sodium hydroxide solution and evaporated to the dry state in a vacuum. The residue is purified on RP-18 (mobile solvent: gradient that consists of water/acetonitrile/tetrahydrofuran).

Yield: 7.65 g (76% of theory) of a colorless, amorphous powder.

Water content: 7.4%

Elementary analysis (relative to anhydrous substance):

Cld: C 44.68 H 4.67 Gd 19.18 N 11.96

Fnd: C 44.80 H 4.78 Gd 19.03 N 12.10

### EXAMPLE 4

N,N-Bis[1,4,7-tris(carboxylatomethyl)-1,4,7,10-tetraazacyclododecane-Gd-complex-10-(3-aza-4-oxo-hexan-5-ylic)-acid]-4,4′-methylene-N′,N′-bis[(3-methoxy)-2-naphtoic acid]-tetrahydrazide

2.12 g (20 mmol) of sodium carbonate is added to 5.03 g (3.07 mmol) of the title compound of Example 3, dissolved in 30 ml of water. 1.01 g (8 mmol) of dimethyl sulfate is added in drops at 0° C., and then it is stirred for 24 hours at room temperature. It is set at pH 7.4 with concentrated hydrochloric acid and evaporated to the dry state in a vacuum. The residue is purified on RP-18 (mobile solvent: gradient that consists of water/acetonitrile/tetrahydrofuran).

Yield: 4.92 g (96% of theory) of an amorphous solid.

Water content: 10.1%

Elementary analysis (relative to anhydrous substance):

Cld: C 45.37 H 4.83 Gd 18.86 N 11.76

Fnd: C 45.51 H 4.94 Gd 19.01 N 11.61

### EXAMPLE 5

N,N-Bis[1,4,7-tris(carboxylatomethyl)-1,4,7,10-tetraazacyclododecane-Gd-complex-10-(3-aza-4-oxo-pentan-5-ylic)-acid]-N′-[(3-hydroxy)-2-naphthoic acid]-dihydrazide

19.56 g (31.76 mmol) of the Gd complex of 10-(4-carboxy-2-oxo-3-aza-butyl)-1,4,7,10-tetraaza-cyclododecane-1,4,7-triacetic acid (Gd-Gly-Me-DOTA), 2.69 g (63.52 mmol) of lithium chloride and 3.66 g (31.76 mmol) of N-hydroxysuccinimide are dissolved in 200 ml of dimethyl sulfoxide while being heated slightly. Then, 6.42 g (31.76 mmol) of 2-hydroxy-3-napthoic acid-hydrazide is added, and it is stirred for 20 minutes at room temperature. It is cooled to 10° C., and 9.83 g (47.64 mmol) of N,N′-dicyclohexylcarbodiimide is added, and it is stirred overnight at room temperature. The reaction solution is poured into a mixture that consists of 1000 ml of acetone/1000 ml of diethyl ether, and the precipitated solid is filtered off. Further purification is carried out by RP-18 chromatography (mobile solvent: gradient that consists of water/acetonitrile/tetrahydrofuran). After the fractions are concentrated by evaporation, 21.59 g (85% of theory) of a colorless, amorphous solid is obtained.

Water content: 7.4%

Elementary analysis (relative to anhydrous substance):

Cld: C 43.55 H 4.54 Gd 19.66 N 12.26

Fnd: C 43.41 H 4.61 Gd 19.50 N 12.40

### EXAMPLE 6

N,N-Bis[1,4,7-tris(carboxylatomethyl)-1,4,7,10-tetraazacyclododecane-Gd-complex-10-(3-aza-4-oxo-pentan-5-ylic)-acid]-N′-[(3-methoxy)-2-napthoic acid]-dihydrazide

2.12 g (20 mmol) of sodium carbonate is added to 4.91 g (6.14 mmol) of the title compound of Example 5, dissolved in 30 ml of water. 1.01 g (8 mmol) of dimethyl sulfate is added in drops at 0° C., and it then is stirred for 24 hours at room temperature. It is set at pH 7.4 with concentrated hydrochloric acid and evaporated to the dry state in a vacuum. The residue is purified on RP-18 (mobile solvent: gradient that consists of water/acetonitrile/tetrahydrofuran).

Yield: 4.85 g (97% of theory) of an amorphous solid.

Water content: 6.8%

Elementary analysis (relative to anhydrous substance):

Cld: C 44.27 H 4.71 Gd 19.32 N 12.05

Fnd: C 44.40 H 4.82 Gd 19.49 N12.16

### EXAMPLE 7

N,N-Bis[1,4,7-tris(carboxylatomethyl)-1,4,7,10-tetraazacyclododecane-Gd-complex-10-(3-aza-4-oxo-pentan-5-ylic]-acid]-4,4-methylene-N′,N′-bis[(3-hydroxy)-2-napthoic acid]-tetrahydrazide

9.83 g (12.29 mmol) of the title compound of Example 5 is refluxed together with 50 ml of acetic acid/50 ml of water, 20 g (243.8 g mmol) of sodium acetate and 0.37 g (12.3 mmol) of formaldehyde (as a 30% aqueous solution) for 3 hours. It is allowed to cool to room temperature, set at pH 7.5 with 10% aqueous sodium hydroxide solution and evaporated to the dry state in a vacuum. The residue is purified on RP-1 8(mobile solvent: gradient that consists of water/acetonitrile/tetrahydrofuran).

Yield: 7.23 g (73% of theory) of a colorless, amorphous powder.

Water content: 8.3%

Elementary analysis (relative to anhydrous substance):

Cld: C 43.97 H 4.50 Gd 19.51 N 12.17

Fnd: C 44.08 H 4.62 Gd 19.67 N 12.28

### EXAMPLE 8

N′,N-Bis[1,4,7-tris(carboxylatomethyl)-1,4,7,10-tetraazacyclododecane-Gd-complex-10-(3-aza-4-oxo-pentan-5-ylic]-acid]-4,4 methylene-N′,N′-bis[(3-methoxy)2-naphthoic acid]-tetrahydrazide

2.12 g (20 mmol) of sodium carbonate is added to 4.95 g (3.07 mmol) of the title compound of Example 7, dissolved in 30 ml of water. 1.01 g (8 mmol) of dimethyl sulfate is added in drops at 0° C., and it then is stirred for 24 hours at room temperature. It is set at pH 7.4 with concentrated hydrochloric acid and evaporated to the dry state in a vacuum. The residue is purified on RP-18 (mobile solvent: gradient that consists of water/acetonitrile/tetrahydrofuran).

Yield: 4.78 g (95% of theory) of an amorphous solid.

Water content: 7.8%

Elementary analysis (relative to anhydrous substance):

Cld: C 44.68 H 4.67 Gd 19.18 N 11.96

Fnd: C 44.75 H 4.80 Gd 19.02 N 12.04

### EXAMPLE 9

N-[1,4,7-Tris(carboxylatomethyl)-1,4,7,10-tetraazacyclododecane-Gd-complex-10-(3-aza-4-oxo-hexan-5-ylic)-acid]-N′-[(3-ethoxy)-2-napthoic acid]-dihydrazide

2.12 g (20 mmol) of sodium carbonate is added to 5 g (6.14 mmol) of the title compound of Example 1, dissolved in 30 ml of water. 1.23 g (8 mmol) of diethyl sulfate is added in drops at 0° C., and then it is stirred for 24 hours at room temperature. It is set at pH 7.4 with concentrated hydrochloric acid and evaporated to the dry state in a vacuum. The residue is purified on RP-18 (mobile solvent: gradient that consists of water/ acetonitrile/tetrahydrofuran).

Yield: 4.89 g (96% of theory) of an amorphous solid.

Water content: 11.1%

Elementary analysis (relative to anhydrous substance):

Cld: C 44.86 H 5.10 Gd 18.95 N 11.81

Fnd: C 44.74 H 5.21 Gd 19.16 N 11.96

### EXAMPLE 10a

2-[4-(3-Oxapropionic acid ethyl ester)]-phenyl-acetic Acid Methyl Ester

233.8 g (1.4 mol) of 2-bromoacetic acid-ethyl ester is added to 200 g (1.204 mol) of 4-hydroxyphenylacetic acid methyl ester, 212 g (2 mmol) of sodium carbonate in 2000 ml of acetone, and it is refluxed for 5 hours. The solid is filtered off, and the filtrate is evaporated to the dry state in a vacuum. The residue is chromatographed on silica gel (mobile solvent:n-hexane/ethyl acetate=15: 1).

Yield: 288.5 g (95% of theory) of a colorless oil.

Elementary analysis:

Cld: C 61.90 H 6.39

Fnd: C 62.05 H 6.51

### EXAMPLE 10b

2-Bromo-2-[4-(oxapropionic acid ethyl ester)]-phenyl-acetic Acid Methyl Ester

201 g (1.13 mol) of N-bromosuccinimide and 100 ml of dibenzoyl peroxide are added to 285 g (1.13 mol) of the title compound of Example 10a, dissolved in 2000 ml of carbon tetrachloride. It is refluxed for 8 hours. It is cooled in an ice bath, the precipitated succinimide is filtered off, and the filtrate is evaporated to the dry state in a vacuum. The residue is purified on silica gel (mobile solvent:n-hexane/acetone=15:1).

Yield: 359.2 g (96% of theory) of a colorless, viscous oil.

Elementary analysis:

Cld: C 47.15 H 4.57 Br 24.16

Fnd: C 47.28 H 4.47 Br 24.30

### EXAMPLE 10c

2-(1,4,7,10-Tetraazacyclododec-1-yl)-2-[4-(3-oxapropionic acid-ethyl ester)]-phenyl-acetic Acid Methyl Ester

350 g (1.057 mol) of the title compound of Example 10b is added to 603 g (3.5 mol) of 1,4,7,10-tetraazacyclododecane in 6000 ml of chloroform, and it is stirred overnight at room temperature. It is extracted 3 times with 3000 ml of water, the organic phase is dried on magnesium sulfate and evaporated to the dry state in a vacuum. The residue is used without further purification in the next stage=>10d.

Yield: 448 g (quantitative) of a viscous, yellowish oil.

Elementary analysis:

Cld: C 59.70 H 8.11 N 13.26

Fnd: C 59.84 H 8.25 N 13.20

### EXAMPLE 10d

2-[1,4,7-Tris(carboxymethyl)-1,4,7,10-tetraazacyclododec-10yl]-2-[4-(3-oxapropionic acid)]-phenyl-acetic Acid

445 g (1.053 mol) of the title compound of Example 10c and 494 g (5.27 mol) of chloroacetic acid are dissolved in 4000 ml of water. It is set at pH 10 with 30% sodium hydroxide solution. It is heated to 70° C., and the pH is kept at 10 by adding 30% sodium hydroxide solution. It is stirred for 8 hours at 70° C. Then, it is set at pH 13 and refluxed for 30 minutes. The solution is cooled in an ice bath and set at pH 1 by adding concentrated hydrochloric acid. It is evaporated to the dry state in a vacuum. The residue is taken up in 4000 ml of methanol, and it is absorptively precipitated for one hour at room temperature. The precipitated common salt is filtered out, the filtrate is evaporated to the dry state, and the residue is purified on RP-18 (mobile solvent: gradient that consists of water/ethano1acetonitrile).

Yield: 403 g (69% of theory) of a colorless solid.

Water content: 10.2%

Elementary analysis (relative to anhydrous substance):

Cld: C 51.98 H 6.18 N 10.10

Fnd: C 52.15 H 6.29 N 10.22

### EXAMPLE 10e

2-[1,4,7-Tris(carboxylatomethyl)-1,4,7,10-tetraazacyclododec-10-yl, Gd-complex]-2-[4-(3-oxapropionic acid)]-phenyl-acetic Acid

130.73 g (360.65 mmol) of gadolinium oxide is added to 400 g (721.3 mmol) of the title compound of Example 1Od in 2000 ml of water, and it is stirred for 5 hours at 90° C. The solution is filtered, and the filtrate is freeze-dried.

Yield: 511 g (quantitative) of an amorphous, colorless powder.

Water content: 11.0%

Elementary analysis (relative to anhydrous substance):

Cld: C 40.67 H 4.41 Gd 22.19 N 7.98

Fnd: C 40.80 H 4.52 Gd 22.03 N 7.78

### EXAMPLE 10f

N-(1,4,7,-Tris(carboxylatomethyl)-1,4,7,10-tetraazacyclododecane-Gd-complex-10-[1-carboxylate-1-[3-oxapropionic acid)-phenyl]-methyl)-N′-[3-(hydroxy)-2-naphthoic acid]-dihydrazide, Sodium SSalt

22.51 g (31.76 mmol) of the title compound of Example 10e, 2.69 g (63.52 mmol) of lithium chloride and 3.66 g (31.76 mmol) of N-hydroxysuccinimide are dissolved in 200 ml of dimethyl sulfoxide while being heated slightly. Then, 6.42 g (31.76 mmol) of 2-hydroxy-3-naphthoic acid hydrazide is added, and it is stirred for 20 minutes at room temperature. It is cooled to 10° C., 9.83 g (47.64 mmol) of N,N′-dicyclohexylcarbodiimide is added, and it is stirred overnight at room temperature. The reaction solution is poured into a mixture that consists of 1000 ml of acetone/1000 ml of diethyl ether, and the deposited solid is filtered off Further purification is carried out by RP-18 chromatography (mobile solvent: gradient that consists of water/acetonitrile/tetrahydrofuran). After the fractions are concentrated by evaporation, it is dissolved in a little water, the pH is set at 7.4 with 2N sodium hydroxide solution and then freeze-dried. 25.0 g (86% of theory) of a colorless, amorphous solid is obtained.

Water content: 10.3%

Elementary analysis (relative to anhydrous substance):

Cld: C 45.95 H 4.19 Gd 17.19 N 9.19 Na 2.51

Fnd: C 46.11 H 4.27 Gd 17.08 N9.27 Na 2.60

### EXAMPLE 11

N-(1,4,7,-Tris(carboxylatomethyl)-1,4,7,10-tetraazacyclododecane-Gd-complex-10-[1-carboxylate-1-[3-oxapropionic acid)-phenyl]-methyl-N′-[3-(methoxy),2-naphthoic acid]-dihydrazide, sodium salt

2.12 g (20 mmol) of sodium carbonate is added to 5.62 g (6.14 mmol) of the title compound of Example lOf, dissolved in 30 ml of water. 1.01 g (8 mmol) of dimethyl sulfate is added in drops at 0° C., and it is then stirred for 24 hours at room temperature. It is set at pH 7.4 with concentrated hydrochloric acid and evaporated to the dry state in a vacuum. The residue is purified on RP-18 (mobile solvent: gradient that consists of water/acetonitrile/tetrahydrofiuran).

Yield: 5.48 g (96% of theory) of an amorphous solid.

Water content: 7.9%

Elementary analysis (relative to anhydrous substance):

Cld: C 46.55 H 4.34 Gd 16.93 N 9.05 Na 2.47

Fnd: C 46.40 H 4.44 Gd 17.07 N 9.15 Na 2.58

### EXAMPLE 12

N-Bis(1,4,7-tris(carboxylatomethyl)-1,4,7,10-tetraazacyclododecane-Gd-complex-10-<1-carboxylate-1-[4-(3-oxapropionic acid]-phenyl>-methyl)-4,4′methylene-N,N-bis[(3-hydroxy)-2-naphthoic acid]-tetrahydrazide, Disodium Salt

11.24 g (12.29 mmol) of the title compound of Example 10f is refluxed together with 50 ml of acetic acid/50 ml of water, 20 g (243.8 g mmol) of sodium acetate and 0.37 g (12.3 rnmol) of formaldehyde (as 30% aqueous solution) for 3 hours. It is allowed to cool to room temperature, set at pH 7.5 with 10% aqueous sodium hydroxide solution and evaporated to the dry state in a vacuum. The residue is purified on RP-18 (mobile solvent: gradient that consists of water/acetonitrile/tetrahydrofuran).

Yield: 8.04 g (71% of theory) of a colorless, amorphous powder.

Water content: 7.3%

Elementary analysis (relative to anhydrous substance):

Cld: C 46.30 H 4.16 Gd 17.07 N 9.13 Na 2.50

Fnd: C 46.46 H 4.24 Gd 17.20 N 9.21 Na 2.61

### EXAMPLE 13

N,N-Bis(1,4,7-tris(carboxylatomethyl)-1,4,7,10-tetraazacyclododecane-Gd-complex-10-<1-carboxylate-1-[4-(3-oxapropionic acid]-phenyl-methyl)-4,4′methylene-N,N-bis[(3-methoxy)-2-naphthoic acid]-tetrahydrazide, Disodium Salt

2.12 g (20 mmol) of sodium carbonate is added to 5.65 g (3.07 mmol) of the title compound of Example 12, dissolved in 30 ml of water. 1.01 g (8 mmol) of dimethylsulfate is added in drops at 0° C. and then stirred for 24 hours at room temperature. It is set at pH 7.4 with concentrated hydrochloric acid and evaporated to the dry state in a vacuum. The residue is purified on RP-18 (mobile solvent: gradient that consists of water/acetonitrile/tetrahydrofuran).

Yield: 5.45 g (95% of theory) of an amorphous solid.

Water content: 9.2%

Elementary analysis (relative to anhydrous substance):

Cld: C 46.89 H 4.31 Gd 16.82 N 8.99 Na 2.46

Fnd: C 47.00 H 4.45 Gd 16.99 N 9.09 Na 2.57

### EXAMPLE 14a

4,4′-Di(Naphth-3-oxyacetic acid methyl ester)

25 g (87.31 mmol) of 1,1′-bi-2-naphthol and 37 g (349 mmol) of sodium carbonate are introduced into 200 mnl of dimethylformamide. 29.38 g (192 mmol) of 2-bromoacetic acid methyl ester is added in drops at 60° C., and it is stirred for 8 hours at 60° C. It is allowed to cool to room temperature, solid is filtered out, and the filtrate is evaporated to the dry state in a vacuum. The residue is chromatographed on silica gel (mobile solvent: dichloromethane/methanol=25: 1).

Yield: 34.95 g (93% of theory) of a colorless, crystalline solid.

Elementary analysis:

Cld: C 72.55 H 5.15

Fnd: C 72.69 H 15.26

### EXAMPLE 14b

4,4′-Di(naphth-3-oxyacetic acid hydrazide)

20 g (46.46 mmol) of the title compound of Example 14a is dissolved in 500 ml of boiling methanol, and 14.9 g (465 mmol) of hydrazine is added at boiling heat. It is refluxed for 6 hours. It is evaporated to the dry state, and the residue is crystallized from a little ethanol.

Yield: 17.0 g (85% of theory) of a colorless, crystalline solid.

Elementary analysis:

Cld: C 66.97 H 5.15 N13.02

Fnd: C 67.18 H 5.22 N 13.16

### EXAMPLE 14c

N,N-Bis[1,4,7-tris(carboxylatomethyl)-1,4,7,10-tetraazacyclododecane-Gd-complex-10-(3-aza-4-oxo-hexan-5-ylic)-acid]4,4′di(naphth-3-oxyacetic acid]-tetrahydrazide

20 g (31.76 mmol) of the Gd complex of 10-(4-carboxy-1-methyl-2-oxo-3-aza-butyl)-1,4,7,10-tetraaza-cyclododecane-1,4,7-triacetic acid (Gd-Gly-Me-DOTA), 2.69 g (63.52 mmol) of lithium chloride and 3.66 g (31.76 mmol) of N-hydroxysuccinimide are dissolved in 200 ml of dimethyl sulfoxide while being heated slightly. Then, 6.80 g (15.8 mmol) of the title compound of Example 14b is added, and it is stirred for 20 minutes at room temperature. It is cooled to 10° C., 9.83 g (47.64 mmol) of N,N′-dicyclohexylcarbodiimide is added, and it is stirred overnight at room temperature. The reaction solution is poured into a mixture that consists of 1000 ml of acetone/1000 ml of diethyl ether, and the precipitated solid is filtered off Further purification is carried out by RP-18 chromatography (mobile solvent: gradient that consists of water/acetonitrile/tetrahydrofuran). After the fractions are concentrated by evaporation, 22.21 g (85% of theory) of a colorless, amorphous solid is obtained.

Water content: 9.2%

Elementary analysis (relative to anhydrous substance):

Cld: C 45.03 H 4.75 Gd 19.02 N 11.86

Fnd: C 45.18 H 4.90 Gd 19.25 N 12.01

### EXAMPLE 15

N,N-Bis[1,4,7-tris(carboxylatomethyl)-1,4,7,10-tetraazacyclododecane-Gd-complex-10-3-aza-4-oxo-pentan-5-ylic)-acid]-4,4′-di(naphth-3-oxyacetic acid]-tetrahydrazide

19.56 g (31.76 mmol) of the Gd complex of 10-(4-carboxy-2-oxo-3-aza-butyl)-1,4,7,10-tetraaza-cyclododecane-1,4,7-triacetic acid (Gd-Gly-Me-DOTA), 2.69 g (63.52 mmol) of lithium chloride and 3.66 g (31.76 mmol) of N-hydroxy-succinimide are dissolved in 200 ml of dimethyl sulfoxide while being heated slightly. Then, 6.8 g (15.8 mmol) of the title compound of Example 14b is added, and it is stirred for 20 minutes at room temperature. It is cooled to 10° C., 9.83 g (47.64 mmol) of N,N′-dicyclohexylcarbodiimide is added, and it is stirred overnight at room temperature. The reaction solution is poured into a mixture that consists of 1000 ml of acetone/1000 ml of diethyl ether, and the precipitated solid is filtered off. Further purification is carried out by RP-18 chromatography (mobile solvent: gradient that consists of water/acetonitrile/tetrahydrofuran). After the fractions are concentrated by evaporation, 22.35 g (87% of theory) of a colorless, amorphous solid is obtained.

Water content: 8.7%

Elementary analysis (relative to anhydrous substance):

Cld: C 44.33 H 4.59 Gd 19.34 N 12.06

Fnd: C 44.17 H 4.66 Gd 19.50 N 12.20

### EXAMPLE 16

N,N-Bis[1,4,7-tris(carboxylatomethyl)-1,4,7,10-tetraazacyclododecane-Gd-complex-10-1-carboxylate-1-[4-(3-oxypropionic acid)]-phenyl-methyl]-4,4′di[naphth-3-(oxyacetic acid)]-tetrahydrazide, Disodium Salt

22.51 g (31.76 mmol) of the title compound of Example 10e, 2.69 g (63.52 mmol) of lithium chloride and 3.66 g (31.76 mmol) of N-hydroxysuccinimide are dissolved in 200 ml of dimethyl sulfoxide while being heated slightly. Then, 6.80 g (15.8 mmol) of 2-hydroxy-3-naphthoic acid hydrazide is added, and it is stirred for 20 minutes at room temperature. It is cooled to 10° C., and 9.83 g (47.64 mmol) of N,N′-dicyclohexylcarbodiimide is added, and it is stirred overnight at room temperature. The reaction solution is poured into a mixture that consists of 1000 ml of acetone/1000 ml of diethyl ether, and the precipitated solid is filtered off. Further purification is carried out by RP-18 chromatography (mobile solvent: gradient that consists of water/acetonitrile/tetrahydrofuran). After the fractions are concentrated by evaporation, it is dissolved in a little water, the pH is set at 7.4 with 2N sodium hydroxide solution and then freeze-dried. 24.93 g (85% of theory) of a colorless, amorphous solid is obtained.

Water content: 10.9%

Elementary analysis (relative to anhydrous substance):

Cld: C 46.60 H 4.24 Gd 16.95 N 3.06 Na 2.48

Fnd: C 46.44 H 4.31 Gd 17.08 N 9.15 Na 2.55

### EXAMPLE 17a

9,9-Bis(4-methoxycarbonylmethoxy)phenyl]-fluorene

30.6 g (87.31 mmol) of 9,9-bis(4-hydroxyphenyl)-fluorene and 37 g (349 mmol) of sodium carbonate are introduced into 200 ml of dimethylformamide. 29.38 g (192 mmol) of 2-bromoacetic acid methyl ester is added in drops at 60° C., and it is stirred for 8 hours at 60° C. It is allowed to cool to room temperature, solid is filtered out, and the filtrate is evaporated to the dry state in a vacuum. The residue is chromatographed on silica gel (mobile solvent: dichloromethane/methanol =25: 1).

Yield: 40.59 g (94% of theory) of a colorless, crystalline solid.

Elementary analysis:

Cld: C 75.29 H 5.30

Fnd: C 75.40 H 5.38

### EXAMPLE 17b

9,9-Bis[4-hydrazinocarboxylmethoxy)-phenyl]-fluorene

23 g (46.46 mmol) of the title compound of Example 17a is dissolved in 500 ml of boiling methanol, and 14.9 g (465 mmol) of hydrazine is added at boiling heat. It is refluxed for 6 hours. It is evaporated to the dry state, and the residue is crystallized from a little ethanol.

Yield: 18.61 g (81% of theory) of a colorless, crystalline solid.

Elementary analysis:

Cld: C 70.43 H 15.30 N 11.33

Fnd: C 70.56 H 15.41 N 11.46

### EXAMPLE 17c

N,N-[9,9-Bis(-phenoxyacetic acid)-fluorene>-NN′-bis[1,4,7-tris(carboxylatomethyl)-1,4,7,10-tetraazacyclododecane-Gd-complex-10-(3-aza-4-oxo-hexan-5-ylic)-acid]-tetrahydrazide 20 g (31.76 mmol) of the Gd complex of 10-(4-carboxy-1-methyl-2-oxo-3-aza-butyl)-1,4,7,10-tetraaza-cyclododecane-1,4,7-triacetic acid (Gd-Gly-Me-DOTA), 2.69 g (63.52 mmol) of lithium chloride and 3.66 g (31.76 mmol) of N-hydroxysuccinimide are dissolved in 200 ml of dimethyl sulfoxide while being heated slightly. Then, 7.81 g (15.8 mmol) of the title compound of Example 17b is added, and it is stirred for 20 minutes at room temperature. It is cooled to 10° C., and 9.83 g (47.64 mmol) of N,N′-dicyclohexylcarbodiimide is added, and it is stirred overnight at room temperature. The reaction solution is poured into a mixture that consists of 1000° ml of acetone/1000 ml of diethyl ether, and the precipitated solid is filtered off. Further purification is carried out by RP-18 chromatography (mobile solvent: gradient that consists of water/acetonitrile/tetrahydrofuran). After the fractions are concentrated by evaporation, 23.34 g (86% of theory) of a colorless, amorphous solid is obtained.

Water content: 9.6%

Elementary analysis (relative to anhydrous substance):

Cld: C 46.84 H 4.81 Gd 28.31 N 11.41

Fnd: C 46.98 H 4.90 Gd 18.17 N11.52

### EXAMPLE 18

N,N-[9,9-Bis(4-phenoxyacetic acid)-fluorene]-N′,N′-bis(1,4,7-tris(carboxylatomethyl)-1,4,7,10-tetraazacyclododecane-Gd-complex-10-<1-carboxylate-1-[4-(3-oxapropionic acid)]-phenyl>-methyl)—tetrahydrazide, Disodium Salt

22.51 g (31.76 mmol) of the title compound of Example 10e, 2.69 g (63.52 mmol) of lithium chloride and 3.66 g (31.76 mmol) of N-hydroxysuccinimide are dissolved in 200 ml of dimethyl sulfoxide while being heated slowly. Then, 7.81 g (15.8 mmol) of the title compound of Example 17b is added, and it is stirred for 20 minutes at room temperature. It is cooled to 10° C., 9.83 g (47.64 mmol) of N,N′-dicyclohexylcarbodiimide is added, and it is stirred overnight at room temperature. The reaction solution is poured into a mixture of 1000 ml of acetone/1000 ml of diethyl ether, and the precipitated solid is filtered off. Further purification is carried out by RP-18 chromatography (mobile solvent: gradient that consists of water/acetonitrile/tetrahydrofuran). After the fractions are concentrated by evaporation, it is dissolved in a little water, the pH is set at 7.4 with 2N sodium hydroxide solution, and then it is freeze-dried. 26.4 g (87% of theory) of a colorless, amorphous solid is obtained.

Water content: 7.9%

Elementary analysis (relative to anhydrous substance):

Cld: C 48.17 H 4.30 Gd 16.38 N8.75 Na 2.39

Fnd: C 48.30 H 4.39 Gd 16.50 N 8.86 Na 2.51

### EXAMPLE 19a

Ethylenediamine-N,N′-bis(2-naphthalenesulfonic acid amide) 166 g (732 mmol) of 2-naphthalenesulfonic acid chloride is added to 20 g (333 mmol) of 1,2-diaminoethane, dissolved in 500 ml of pyridine, at 0° C. It is stirred for 6 hours at 0° C. The solution is poured into 2000 ml of water, and the precipitated solid is filtered off. The solid is recrystallized from a little methanol.

Yield: 127.9 g (94% of theory) of a crystalline solid.

Elementary analysis:

Cld: C 64.69 H 4.93 N 6.86 S 7.85 Fnd: C 64.54 H 5.02 N 6.98 S 7.99

### EXAMPLE 19b

3,6-Diaza-3,6-bis(2-naphthalenesulfonyl)-octane-1,8-dicarboxylic Acid Dimethyl Ester

30 g (73.44 mmol) of the title compound of Example 19a and 40.63 g (294 mmol) of potassium carbonate are introduced into 200 ml of dimethylformamide. 24.78 g (162 mmol) of 2-bromoacetic acid methyl ester is added in drops at 60° C., and it is stirred for 8 hours at 60° C. It is allowed to cool to room temperature, solid is filtered out, and the filtrate is evaporated to the dry state in a vacuum. The residue is chromatographed on silica gel (mobile solvent:dichloromethane/methanol=25:1).

Yield: 37.36 g (87% of theory) of a colorless, crystalline solid.

Elementary analysis:

Cld: C 57.52 H 4.83 N4.79 S 10.97

Fnd: C 57.66 H 4.92 N4.68 S 11.08

### EXAMPLE 19c

3,6-Diaza-3,6-bis(2-naphthalenesulfonyl)-octane-1,8-dicarboxylic acid-bis hydrazide

27.16 g (46.46 mmol) of the title compound of Example 17a is dissolved in 500 ml of boiling methanol, and 14.9 g (465 mmol) of hydrazine is added at boiling heat. It is refluxed for 6 hours. It is evaporated to the dry state, and the residue is crystallized from a little ethanol.

Yield: 23.1 g (85% of theory) of a colorless, crystalline solid.

Elementary analysis:

Cld: C 53.41 H 4.83 N 14.37 S 10.97

Fnd: C 53.54 H 4.94 N 14.51 S 11.13

### EXAMPLE 19d

N,N-[3,6-Diaza-3,6-bis(2-naphthalenesulfonyl)-octane-1,8-dicarboxylic acid]-N′,N′-bis[1,4,7-tris(carboxylatomethyl)-1,4,7,10-tetraazacyclododecane-Gd-complex-10-(3-aza-4-oxo-hexan-5 -ylic)-acid]-tetrahydrazide

20 g (31.76 mmol) of the Gd complex of 10-(4-carboxy-1-methyl-2-oxo-3-aza-butyl)-1,4,7,10-tetraaza-cyclododecane-1,4,7-triacetic acid (Gd-Gly-Me-DOTA), 2.69 g (63.52 mmol) of lithium chloride and 3.66 g (31.76 mmol) of N-hydroxysuccinimide are dissolved in 200 ml of dimethyl sulfoxide while being heated slightly. Then, 9.24 g (15.8 mmol) of the title compound of Example 19c is added, and it is stirred for 20 minutes at room temperature. It is cooled to 10° C., 9.83 g (47.64 mmol) of N,N′-dicyclohexylcarbodiimide is added, and it is stirred overnight at room temperature. The reaction solution is poured into a mixture that consists of 1000° ml of acetone/1000 ml of diethyl ether, and the precipitated solid is filtered off. Further purification is carried out by RP-18 chromatography (mobile solvent: gradient that consists of water/acetonitrile/tetrahydrofuran). After the fractions are concentrated by evaporation, 24.85 g (87% of theory) of a colorless, amorphous solid is obtained.

Water content: 9.8%

Elementary analysis (relative to anhydrous substance):

Cld: C 42.51 H 4.68 Gd 17.39 N 12.39 S 3.55

Fnd: C 42.37 H 4.75 Gd 17.53 N 12.50 S 3.44

### EXAMPLE 20

N,N-[3,6-Diaza-3,6-bis(2-naphthalenesulfonyl)-octane-1,8-dicarboxylic acid]-N′,N′-bis[1,4,7-tris(carboxylatomethyl)-1,4,7,10-tetraaza-cyclododecane-Gd-complex-10-<1-carboxylate-1-[4-(3-oxapropionic acid)]-phenyl>-methyl)-tetrahydrazide, Disodium Salt

22.51 g (31.76 mmol) of the title compound of Example 10e, 2.69 g (63.52 mmol) of lithium chloride, and 3.66 g (31.76 mmol) of N-hydroxysuccinimide are dissolved in 200 ml of dimethyl sulfoxide while being heated slightly. Then, 9.24 g (15.8 mmol) of the title compound of Example 19c is added, and it is stirred for 20 minutes at room temperature. It is cooled to 10° C., 9.83 g (47.64 mmol) of N,N′-dicyclohexylcarbodiimide is added, and it is stirred overnight at room temperature. The reaction solution is poured into a mixture that consists of 1000 ml of acetone/i000 ml of diethyl ether, and the precipitated solid is filtered off. Further purification is carried out by RP-18 chromatography (mobile solvent: gradient that consists of water/acetonitrile/tetrahydrofuran). After the fractions are concentrated by evaporation, it is dissolved in a little water, the pH is set at 7.4 with 2N sodium hydroxide solution, and then it is freeze-dried. 27.31 g (86% of theory) of a colorless, amorphous solid is obtained.

Water content: 10.1%

Elementary analysis (relative to anhydrous substance):

Cld: C 44.22 H 4.21 Gd 15.65 N 9.76 Na 2.29 S 3.19

Fnd: C 44.35 H 4.32 Gd 15.71 N9.89 Na 2.38 S 3.31

### EXAMPLE 21

a) 2-Methoxy-6-[(4-methoxycarbonyl)cyclohexylcarbonyl]-naphthalene

15.72 g (0.1 mol) of 2-methoxynaphthalene is dissolved under nitrogen in 130 ml of nitrobenzene, cooled to 12° C. and mixed with 17.3 g (0.13 mol) of aluminum chloride. Then, 26.6 g (0.13 mol) of 4-chlorocarbonyl-cyclohexanecarboxylic acid-methyl ester (Calaminus, W. et al., Z. Naturforsch. B, 41, 1011-1014 (1986)), dissolved in nitrobenzene, is slowly added in drops at 12° C., and it is stirred for 2 hours at this temperature and overnight at room temperature. The mixture is added to ice-cold concentrated hydrochloric acid and extracted several times with dichloromethane. The combined organic phase is dried on sodium sulfate, and evaporated to the dry state in a vacuum. The residue is chromatographed on silica gel (mobile solvent:hexane/ethyl acetate=3:1).

Yield: 21.2 g (65% of theory) of a colorless solid

Elementary analysis:

Cld: C 73.60 H 6.79

Fnd: C 73.42 H 6.91

b) 2-Methoxy-6-[(4-carboxy)cyclohexylcarbonyl]-naphthalene

20.4 g (62.5 mmol) of the methyl ester that is described in Example 21a above is dissolved in dioxane, and after 250 ml of 2N NaOH is added, it is stirred overnight at room temperature. Then, the solution is evaporated to the dry state, and the residue is dispersed between ethyl acetate and dilute hydrochloric acid. The aqueous phase is extracted several times with ethyl acetate, and the combined organic phase is dried on sodium sulfate.

Yield: 20.3 g (quantitative)

Elementary analysis:

Cld: C 73.06 H 6.45

Fnd: C 72.92 H 6.61

c) 2-Methoxy-6-[(4-[2-benzyloxycarbonylaminoethyl]-carbamoyl)cyclohexylcarbonyl]-naphthalene

15.62 g (50 mmol) of the carboxylic acid that is described in Example 21b above is dissolved in tetrahydrofuran (THF), and after 20° ml of triethylamine in acetone/ice bath is added, it is cooled to −15° C. At this temperature, 6.43 ml (50 mmol) of isobutyl chloroformate is added in drops, and it is stirred for 15 minutes.

In another vessel, 15.0 g (65 mmol) of N-(benzyloxycarbonyl)ethylenediamine hydrochloride (Eisenbrand, G. et al., Synthesis 1996, 1246-1258) is simultaneously taken up in dichloromethane, shaken out twice with dilute sodium hydroxide solution, the organic phase is dried on sodium sulfate and evaporated to the dry state. The remaining oil is dissolved in THF and added in drops to the above-described reaction at −15° C. It is allowed to stir for 2 more hours at this temperature and overnight at room temperature. Then, the suspension is evaporated to the dry state, and the residue is dispersed between dichloromethane and water. The organic phase is washed in succession with dilute hydrochloric acid and with sodium carbonate solution and dried on sodium sulfate. After concentration by evaporation in vacuo, it is recrystallized from hexane.

Yield: 20.3 g (83% of theory) of a colorless solid

Elementary analysis:

Cld: C 71.29 H 6.60 N5.73

Fnd: C 71.13 H 6.88 N5.67

d) para-Nitrophenyl-active ester of the gadolinium complex of 10-[4-carboxy-1-methyl-2-oxo-3-azabutyl]-1,4,7,10-tetraazacyclododecane-1,4,7-triacetic Acid

10.0 g (15.9 mmol) of the gadolinium complex of 10-[4-carboxy-1-methyl-2-oxo-3-azabutyl]-1,4,7,10-tetraazacyclododecane-1,4,7-triacetic acid (DE 196 52 386, Schering A G, (priority: 12/4/96)) is dissolved in 70 ml of water, mixed with 1.28° ml (15.9 mmol) of pyridine, and the solution is freeze-dried. The lyophilizate that is obtained is taken up in 120 ml of pyridine, mixed with 7.24 g (23.8 mmol) of bis-(4-nitrophenyl)carbonate, and the suspension is stirred for 3 days at room temperature. Then, the solid is suctioned off, washed with pyridine and dichloromethane and dried at 40° C. in a vacuum.

Yield: 11.47 g (96.2% of theory)

Elementary analysis:

Cld: C 39.99 H 4.43 N 11.19 Gd 20.94

Fnd: C 39.71 H 4.66 N 11.01 Gd 20.32

e) Amide conjugate of the gadolinium complex of 10-[4-carboxy-1-methyl-2-oxo-3-azabutyl]-1,4,7,10-tetraazacyclododecane-1,4,7-triacetic acid] and 2-methoxy-6-[(4-[2-(aminoethyl]-carbamoyl)cyclohexylcarbonyl]-naphthalene

2.44 g (5 mrnol) of the protected amine that is described in Example 21 c is dissolved in 25 ml of HBr/glacial acetic acid, and it is stirred for 30 minutes at room temperature. After 200 ml of ether is added, the suspension that is produced is stirred overnight, the precipitate is filtered off, washed with ether and then dried in a high vacuum. The hygroscopic hydrobromide is further reacted without additional purification (2.18 g, quantitative).

3 ml of triethylamine and then 2.18 g (5 mmol) of the amine-hydrobroride are added to 5.63 g (7.5 mmol) of the Gd-complex active ester, described in Example 22d above, in 75 ml of DMF. The suspension is stirred overnight at room temperature and then evaporated to the dry state. The residue is dissolved in water and absorptively precipitated with activated carbon for one hour. Carbon is filtered out, and it is ultrafiltered to remove low-molecular components with an Amicon(P) ultrafiltration membrane YC 05 (cut-off: 500 Da). The residue is chromatographed on silica gel (mobile solvent:methanol/water=2:1). The combined fractions are concentrated by evaporation, taken up in water and freeze-dried.

Yield: 3.63 g (69% of theory) of a colorless powder.

Water content (Karl.Fischer): 8.3%

Elementary analysis (relative to anhydrous substance):

Cld: C 49.73 H 5.63 N 10.15 Gd 16.28

Fnd: C 49.52 H 5.88 N 10.09 Gd 15.93

### EXAMPLE 22

a) N_{α}[4-(2-Methoxynaphth-6-oyl)]cyclohexylcarbonyl)-N_{ξ}(t-butoxycarbonyl)-lysine t-butylester

15.62 g (50 mmol) of the carboxylic acid that is described in Example 21b is dissolved in THF, and after 20 ml of triethylamine in acetone/ice bath is added, it is cooled to −15° C. 6.43 ml (50 mmol) of isobutyl chloroformate is added in drops at this temperature and stirred for 15 minutes. 16.63 g (55 mmol) of H-Lys(Boc)-OtBu (Wakimasu, M. et al, Chem. Pharm. Bull. 29, 2592-2597 (1981)) in the least possible amount of THF is added in drops to this suspension at −15° C. It is allowed to stir for 2 more hours at this temperature and overnight at room temperature. Then, the suspension is evaporated to the dry state, and the residue is dispersed between dichloromethane and water. The organic phase is washed in succession with dilute hydrochloric acid and with sodium carbonate solution and dried on sodium sulfate. After concentration by evaporation in vacuo, the residue is chromatographed on silica gel (mobile solvent:ethyl acetate/ethanol=9:1).

Yield: 23.9 g (81% of theory) of a colorless solid

Elementary analysis:

Cld: C 69.13 H 7.17 N 4.74

Fnd: C 68.97 H 7.33 N 4.67

b) N_{α}[4-(2-methoxynaphth-6-oyl)]cyclohexylcarbonyl)-lysine

2.95 g (5 mmol) of the protected amino acid that is described in Example 22a above is suspended in 30 ml of trifluoroacetic acid, and it is stirred for 2 hours at room temperature. After 200 ml of ether is added, the suspension is stirred overnight, the precipitate is filtered off, washed carefully with ether and then dried in a high vacuum to a constant weight in the presence of KOH.

Yield: 2.2 g (quantitative) of a colorless solid

Elementary analysis:

Cld: C 68.16 H 7.32 N 6.36

Fnd: C 68.30 N 7.13 N 6.16

c) N_{ξ}-Lysinamide conjugate of the gadolinium complex of 10-[4-carboxy-1-methyl-2-oxo-3-azabutyl]-1,4,7,10-tetraazacyclododecane-1,4,7-triacetic acid] and N,[4-(2-methoxynaphth-6-oyl)]cyclohexylcarbonyl)-lysine

3 ml of triethylamine and then 2.2 g (5 mmol) of the lysine derivative that is described in Example 22b above are added to 5.63 g (7.5 mmol) of the Gd complex active ester, described in Example 21 d, in 75 ml of DMF. The suspension is stirred overnight at room temperature and then evaporated to the dry state. The residue is dissolved in water and absorptively precipitated for one hour with activated carbon. Carbon is filtered out and ultrafiltered to remove low-molecular components with an Amicon® ultrafiltration membrane YC 05 (cut-off: 500 Da). The residue is chromatographed on silica gel (mobile solvent: methanol/water=2:1). The combined fractions are concentrated by evaporation, taken up in water and freeze-dried.

Yield: 4.0 g (72% of theory) of a colorless powder.

Water content (Karl.Fischer): 5.3%

Elementary analysis (relative to anhydrous substance):

Cld: C 50.22 H 5.75 N 9.32 Gd 14.94

Fnd: C 50.13 H 5.89 N 9.67 Gd 14.41

### EXAMPLE 23

a) 2-Methoxy-6-[(4-methoxycarbonyl)benzoyl]-naphthalene

1.57 g (10 mmol) of 2-methoxynaphthalene is dissolved under nitrogen in 15 ml of nitrobenzene, and it is mixed with 2.88 g (13 mmol) of indium chloride. Then, 2.6 g (13 mmol) of 4-chlorocarbonyl-benzoic acid-methyl ester (Vulakh, E. et al., J. Org. Chem. USSR (Engl. Transl.), 22, 620-627 (1986)), dissolved in nitrobenzene, is slowly added in drops and stirred for 4 hours at 50° C. and overnight at room temperature. The mixture is added to ice-cold concentrated hydrochloric acid and extracted several times with dichloromethane. The combined organic phase is dried on sodium sulfate and evaporated to the dry state in a vacuum. The residue is chromatographed on silica gel (mobile solvent:hexane/ethyl acetate=3:1).

Yield: 1.95 g (61% of theory) of a colorless solid

Elementary analysis:

Cld: C 74.99 H 5.03

Fnd: C 74.62 H 4.91

b) 2-Methoxy-6-[(4-carboxy)benzoyl]-naphthalene

2.0 g (6.25 mmol) of the methyl ester that is described in Example 23a above is dissolved in dioxane, and after 25 ml of 2N NaoH is added, it is stirred overnight at room temperature. Then, the solution is evaporated to the dry state, and the residue is dispersed between ethyl acetate and dilute hydrochloric acid. The aqueous phase is extracted several times with ethyl acetate, and the combined organic phase is dried on sodium sulfate.

Yield: 1.9 g (quantitative)

Elementary analysis:

Cld: C 74.50 H 4.61

Fnd: C 74.32 H 4.60

c) 2-Methoxy-6-[(4-[2-benzyloxycarbonylaminoethyl]-carbamoyl)benzoyl]-naphthalene

1.53 g (5 mmol) of the carboxylic acid that is described in Example 23b above is dissolved in THF, and after 2 ml of triethylamine in acetone/ice bath is added, it is cooled to −15° C. 0.64 ml (5 mmol) of isobutyl chloroformate is added in drops at this temperature and stirred for 15 minutes. In another vessel, 1.5 g (6.5 mmol) of N-(benzyloxy-carbonyl) ethylenediamine hydrochloride (Eisenbrand, G. et al., Synthesis 1996, 1246-1258) in dichloromethane is taken up at the same time, shaken out twice with dilute sodium hydroxide solution, the organic phase is dried on sodium sulfate and evaporated to the dry state. The remaining oil is dissolved in THF and added in drops to the above-described reaction at −15° C. It is allowed to stir for 2 more hours at this temperature and overnight at room temperature. Then, the suspension is evaporated to the dry state, and the residue is dispersed between dichloromethane and water. The organic phase is washed in succession with dilute hydrochloric acid and with sodium carbonate solution, and it is dried on sodium sulfate. After concentration by evaporation in vacuo, it is recrystallized from hexane.

Yield: 1.94 g (80% of theory) of a colorless solid

Elementary analysis:

Cld: C 71.88 H 5.82 N 5.78

Fnd: C 71.70 H 5.94 N 5.66

d) Amide conjugate of the gadolinium complex of 10-[4-carboxy-1-methyl-2-oxo-3-azabutyl]-1,4,7,10-tetraazacyclododecane-1,4,7-triacetic acid] and 2-methoxy-6-[(4-[2-aminoethyl]-carbamoyl)benzoyl]-naphthalene

2.42 g (5 mmol) of the protected amine that is described in Example 23c above is dissolved in 3 ml of BBr/glacial acetic acid, and it is stirred for 30 minutes at room temperature. After 50 ml of ether is added, the suspension that is produced is stirred overnight, the precipitate is filtered off, washed with ether and then dried in a high vacuum. The hygroscopic hydrobromide is further reacted without additional purification (2.16 g, quantitative). 3 ml of triethylamine and then 2.16 g (5 mmol) of the amine-hydrobromide are added to 5.63 g (7.5 mmol) of the Gd-complex active ester in 75 ml of DMF that is described in Example 21d. The suspension is stirred overnight at room temperature and then evaporated to the dry state. The residue is dissolved in water and absorptively precipitated for one hour with activated carbon. Carbon is filtered out and ultrafiltered with an Amicon® ultrafiltration membrane YC 05 (cut-off: 500 Da) to remove low-molecular components. The residue is chromatographed on silica gel (mobile solvent:methano/water=2:1). The combined fractions are concentrated by evaporation, taken up in water and freeze-dried.

Yield: 3.43 g (65% of theory) of a colorless powder.

Water content (Karl.Fischer): 9.1%

Elementary analysis (relative to anhydrous substance):

Cld: C 50.04 H 5.04 N 10.21 Gd 16.38

Fnd: C 49.79 H 4.88 N 10.48 Gd 15.99

### EXAMPLE 24

a) N_{α}[4-(2-Methoxynaphth-6-oyl)]benzoyl)-N(t-butoxycarbonyl)-lysine t-butylester

15.31 g (50 mmol) of the carboxylic acid that is described in Example 23b is dissolved in THF, and after 20 ml of triethylamine in acetone/ice bath is added, it is cooled to −15° C. At this temperature, 6.43 ml (50 mmol) of isobutyl chloroformate is added in drops and stirred for 15 minutes. 16.63 g (55 mmol) of H-Lys(Boc)-OtBu (Wakimasu, M. et al., Chem. Pharm. Bull. 29, 2592-2597 (1981)) in the least possible amount of THF is added in drops to this suspension at −15° C. It is allowed to stir for 2 more hours at this temperature and overnight at room temperature. Then, the suspension is evaporated to the dry state, and the residue is dispersed between dichloromethane and water. The organic phase is washed in succession with dilute hydrochloric acid and with sodium carbonate solution, and it is dried on sodium sulfate. After concentration by evaporation in vacuo, the residue is chromatographed on silica gel (mobile solvent:ethyl acetate/ethanol=9:1).

Yield: 24.64 g (84% of theory) of a colorless solid

Elementary analysis:

Cld: C 69.61 H 6.53 N 4.77

Fnd: C 69.40 H 6.31 N 4.67

b) N_{α}[4-(2-Methoxynaphth-6-oyl)benzoyl]-lysine

2.93 g (5 mmol) of the protected amino acid that is described in Example 24a above is suspended in 30 ml of trifluoroacetic acid and stirred for 2 hours at room temperature. After 200 ml of ether is added, the suspension is stirred overnight, the precipitate is filtered off, washed carefully with ether and then dried in a high vacuum to a constant weight in the presence of KOH.

Yield: 2.2 q (quantitative) of a colorless solid

Elementary analysis:

Cld: C 68.79 H 6.47 N 6.42

Fnd: C 68.60 H 6.77 N 6.48

c) N-Lysinamide conjugate of the gadolinium complex of 10-[4-carboxy-1-methyl-2-oxo-3-azabutyl]-1,4,7,10-tetraazacyclododecane-1,4,7-triacetic acid] and N_{α}[4-(2-methoxynaphth-6-oyl)benzoyl]-lysine

3 ml of triethylamine and then 2.2 g (5 mmol) of the lysine derivative that is described in Example 24b above are added to 5.63 g (7.5 mmol) of the Gd complex-active ester, described in Example 21d, in 75 ml of DMF. The suspension is stirred overnight at room temperature and then evaporated to the dry state. The residue is dissolved in water and absorptively precipitated for one hour with activated carbon. Carbon is filtered out and ultrafiltered with an Axicon® ultrafiltration membrane YC 05 (cut-off. 500 Da) to remove low-molecular components. The residue is chromatographed on silica gel (mobile solvent: methanoVwater =2: 1). The combined fractions are concentrated by evaporation, taken up in water and freeze-dried.

Yield: 3.96 g (70% of theory) of a colorless powder.

Water content (Karl.Fischer): 7.3%

Elementary analysis (relative to anhydrous substance):

Cld: C 50.42 H 5.38 N 9.35 Gd 15.00

Fnd: C 50.48 H 5.20 N 9.61 Gd 14.64

### EXAMPLE 25

a) 2-Methoxy-6-[(4-methoxycarbonyl)naphth-1-oyl]-naphthalene

1.57 g (10 mmol) of 2-methoxynaphthalene is dissolved under nitrogen in 15 ml of nitrobenzene and mixed with 2.88 g (13 mmol) of indium chloride. Then, 3.23 g (13 mmol) of 1,4-naphthalene-dicarboxylic acid-monochloride-monomethyl ester (Frischkorn, Hans et al., Ger. Offen. (1978), DE 2715567), dissolved in nitrobenzene, is slowly added in drops, and it is stirred for 4 hours at 50° C. and overnight at room temperature. The mixture is added to ice-cold concentrated hydrochloric acid, and it is extracted several times with dichloromethane. The combined organic phase is dried on sodium sulfate and evaporated to the dry state in a vacuum. The residue is chromatographed on silica gel (mobile solvent:hexane/ethyl acetate=4:1).

Yield: 2.53 g (68% of theory) of a colorless solid

Elementary analysis:

Cld: C 77.40 H 5.41

Fnd: C 77.10 H 15.33

b) 2-Methoxy-6-[(4-carboxy)naphth-1-oyl]-naphthalene

2.33 g (6.25 mmol) of the methyl ester that is described in Example 25a above is dissolved in dioxane and, after 25 ml of 2N NaOH is added, it is stirred overnight at room temperature. Then, the solution is evaporated to the dry state, and the residue is dispersed between ethyl acetate and dilute hydrochloric acid. The aqueous phase is extracted several times with ethyl acetate, and the combined organic phase is dried on sodium sulfate.

Yield: 2.2 g (quantitative)

Elementary analysis:

Cld: C 77.08 H 5.06

Fnd: C 76.90 H 5.31

c) 2-Methoxy-6-([4-[2-benzyloxycarbonylaminoethy]-carbamoyl)naphth-1-oyl]-naphthalene

1.79 g (5 mmol) of the carboxylic acid that is described in Example 25b above is dissolved in THF, and after 2 ml of triethylamine is added, it is cooled in acetone/ice bath to −15° C. At this temperature, 0.64 ml (5 mmol) of isobutyl chloroformate is added in drops, and it is stirred for 15 minutes. In another vessel, 1.5 g (6.5 mmol) of N-(benzyloxycarbonyl)ethylenediamine hydrochloride (Eisenbrand, G. et al., Synthesis 1996, 1246-1258) in dichloromethane is taken up at the same time, shaken out twice with dilute sodium hydroxide solution, the organic phase is dried on sodium sulfate and evaporated to the dry state. The remaining oil is dissolved in THF and added in drops to the above-described reaction at −15° C. It is allowed to stir for 2 more hours at this temperature and overnight at room temperature. Then, the suspension is evaporated to the dry state, and the residue is dispersed between dichloromethane and water. The organic phase is washed in succession with dilute hydrochloric acid and with sodium carbonate solution, and it is dried on sodium sulfate. After concentration by evaporation in vacuo, it is recrystallized from hexane.

Yield: 2.36 g (88% of theory) of a colorless solid

Elementary analysis:

Cld: C 73.86 H 6.01 N 5.22

Fnd: C 73.70 H 5.94 N 5.47

d) Amide conjugate of the gadolinium complex of 10-[4-carboxy-1-methyl-2-oxo-3-azabutyl]-1,4,7,10-tetraazacyclododecane-1,4,7-triacetic acid] and 2-methoxy-6-[(4-[2-aminoethyl]-carbamoyl)naphth-1-oyl]-naphthalene

2.68 g (5 mmol) of the protected amine that is described in Example 25c above is dissolved in 3 ml of HBr/glacial acetic acid, and it is stirred for 30 minutes at room temperature. After 50 ml of ether is added, the suspension that is produced is stirred overnight, the precipitate is filtered off, washed with ether and then dried in a high vacuum. The hygroscopic hydrobromide is further reacted without additional purification (2.42 g, quantitative). 3 ml of triethylamine and then 2.42 g (5 mmol) of the amine-hydrobromide are added to 5.63 g (7.5 mmol) of the Gd complex, described in Example 22d, in 75 ml of DMF. The suspension is stirred overnight at room temperature and then evaporated to the dry state. The residue is dissolved in water and absorptively precipitated for one hour with activated carbon. Carbon is filtered out and ultrafiltered with an Amicon(R) ultrafiltration membrane YC OS (cut-off: 500 Da) to remove low-molecular components. The residue is chromatographed on silica gel (mobile solvent:methanol/water=3:1). The combined fractions are concentrated by evaporation, taken up in water and freeze-dried.

Yield: 3.81 g (69% of theory) of a colorless powder.

Water content (Karl.Fischer): 8.3%

Elementary analysis (relative to anhydrous substance):

Cld: C 52.21 H 5.18 N 9.69 Gd 15.54

Fnd: C 51.94 H 5.11 N 9.67 Gd 15.28

### EXAMPLE 26

a) 2-Dimethylamino-6-[(4-carboxy)cyclohexylcarbonyl]-naphthalene

7.81 g (25 mmol) of the carboxylic acid that is described in Example 21b is dissolved in THF, and after 5.1 g (100 mmol) of lithium-dimethylamide (Aldrich) is added, it is refluxed for 8 hours and then stirred overnight at room temperature. Then, it is evaporated to the dry state, the residue is dispersed between ethyl acetate and aqueous citric acid solution, and the organic phase is finally washed with water and dried on sodium sulfate. The crude product is chromatographed on silica gel (mobile solvent:diisopropyl ether/glacial acetic acid=19:1).

Yield: 3.66 g (45% of theory)

Elementary analysis (relative to anhydrous substance):

Cld: C 73.82 H 7.12 N 4.30

Fnd: C 74.10 H 6.88 N 4.07

b) N_{α}[4-(2-Dimethylamino-naphth-6-oyl)]cyclohexylcarbonyl)-N_{ξ}(benzyloxycarbonyl)-lysine Methylester

1.63 g (5 mmol) of the carboxylic acid that is described in Example 26a above is dissolved in TUF, and after 2 ml of triethylamine in acetone/ice bath is added, it is cooled to −15° C. At this temperature, 0.64 ml (5 mmol) of isobutyl chloroformate is added in drops, and it is stirred for 15 minutes. 1.62 g (5.5 mmol) of H-Lys(Z)-OMe (Slotin, L. A. et al., Can. J. Chem. 55, 4257-66 (1977)) in the smallest possible amount of THF is added in drops to this suspension at −15° C. It is allowed to stir for 2 more hours at this temperature and overnight at room temperature. Then, the suspension is evaporated to the dry state, and the residue is dispersed between dichloromethane and water. The organic phase is washed in succession with dilute hydrochloric acid and with sodium carbonate solution, and it is dried on sodium sulfate. After concentration by evaporation in vacuo, the residue is chromatographed on silica gel (mobile solvent:ethyl acetate/ethanol=9:1).

Yield: 2.59 g (86% of theory)

Elementary analysis:

Cld: C 69.86 H 7.20 N 6.98

Fnd: C 69.91 H 7.44 N 6.67

c) N_{ξ}-Lysinamide conjugate of the gadolinium complex of 10-[4-carboxy-1-methyl-2-oxo-3-azabutyl]-1,4,7,10-tetraazacyclododecane-1,4,7-triacetic acid] and N,[4-(2-dimethylamino-naphth-6-oyl)] cyclohexylcarbonyl)-lysine

3.01 g (5 mmol) of the protected amino acid that is described in Example 26b above is dissolved in 25 ml of HBr/glacial acetic acid, and it is stirred for 30 minutes at room temperature. After 200 ml of ether is added, the suspension that is produced is stirred overnight, the precipitate is filtered off, washed with ether and dried in a vacuum. The hydrobromide is then dissolved in methanol, mixed with 25 ml of 2N sodium hydroxide solution and stirred overnight at room temperature. Then, the organic solvent is concentrated by evaporation, the aqueous solution is set at pH 8.5 with dilute hydrochloric acid, and it is evaporated to the dry state. The N_{α}[4-(2-dimethylamino-naphth-6-oyl)]-cyclohexylcarbonyl)-lysine that is produced is reacted without further purification (2.3 g, quantitative).

3 ml of triethylamine and then 2.3 g (5 mmol) of the described lysine derivative are added to 5.63 g (7.5 mmol) of the Gd complex active ester, described in Example 21d, in 75 ml of DMF. The suspension is stirred overnight at room temperature, and then it is evaporated to the dry state. The residue is dissolved in water and absorptively precipitated for one hour with activated carbon. Carbon is filtered out, and it is ultrafiltered with an Amicon® ultrafiltration membrane YC 05 (cut-off: 500 Da). The residue is chromatographed on silica gel (mobile solvent:methanol/water=2:1). The combined fractions are concentrated by evaporation and taken up in a little water. The solution is mixed with 4 ml of anion exchanger IRA 410 (OH⁻-form), filtered, and the filtrate is freeze-dried.

Yield: 4.1 g (72% of theory)

Water content (Karl.Fischer): 7.3%

Elementary analysis (relative to anhydrous substance):

Cld: C 50.74 H 5.96 N 10.52 Gd 14.76

Fnd: C 50.43 H 5.96 N 10.67 Gd 14.21

### EXAMPLE 27

a) N_{α}[4-(2-Methoxynaphth-6-oyl)naphth-1-oyl]-N_{ξ}(t-butoxycarbonyl)-lysinet-butylester

17.92 g (50 mmol) of the carboxylic acid that is described in Example 25b is dissolved in THF, and after 20 ml of triethylamine in an acetone/ice bath is added, it is cooled to −15° C. At this temperature, 6.43 ml (50 mmol) of isobutyl chloroformate is added in drops, and it is stirred for 15 minutes. 16.63 g (55 mmol) of H-Lys(Boc)-OtBu (Wakimasu, M. et al., Chem. Pharm. Bull. 29, 2592-2597 (1981)) in the least possible amount of TII is added in drops to this suspension at −15° C. It is allowed to stir for 2 more hours at this temperature and overnight at room temperature. Then, the suspension is evaporated to the dry state, and the residue between dichloromethane and water is dispersed. The organic phase is washed in succession with dilute hydrochloric acid and with sodium carbonate solution, and it is dried on sodium sulfate. After concentration by evaporation in vacuo, the residue is chromatographed on silica gel (mobile solvent:ethyl acetate/ethanol=10:1).

Yield: 31.94 g (79% of theory) of a colorless solid

Elementary analysis:

Cld: C 71.45 H 6.63 N 4.39

Fnd: C 71.10 H 6.39 N 4.66

b) N_{α}[4-(2-methoxynaphth-6-oyl)naphth-1-oyl]-lysine

3.19 g (5 mmol) of the protected amino acid that is described in Example 27a above is suspended in 30 ml of trifluoroacetic acid, and it is stirred for 2 hours at room temperature. After 200 ml of ether is added, the suspension is stirred overnight, the precipitate is filtered off, washed carefully with ether and then dried in a high vacuum to a constant weight in the presence of KOH.

Yield: 2.5 g (quantitative) of a colorless solid

Elementary analysis:

Cld: C 71.29 H 6.60 N 5.73

Fnd: C 71.21 H 6.77 N 5.48

c) N_{ξ}-Lysinamide Conjugate of the Gadolinium Complex of 10-[4-carboxy-1-methyl-2-oxo-3-azabutyl]-1,4,7,10-tetraazacyclododecane-1,4,7-triacetic acid] and N_{α}[4-(2-methoxynaphth-6-oyl)naphth-1-oyl]-lysine

3 ml of triethylamine and then 2.5 g (5 mmol) of the lysine derivative that is described in Example 27b above are added to 5.63 g (7.5 mmol) of the Gd complex active ester that is described in Example 21d. The suspension is stirred overnight at room temperature and then evaporated to the dry state. The residue is dissolved in water, and absorptively precipitated for one hour with activated carbon. Carbon is filtered out and ultrafiltered with an Amicon®-ultrafiltration membrane YC 05 (cut-off: 500 Da). The residue is chromatographed on silica gel (mobile solvent:methanol/water=3:1). The combined fractions are concentrated by evaporation, taken up in water and freeze-dried.

Yield: 5.0 g (83% of theory) of a colorless powder.

Water content (Karl.Fischer): 8.9%

Elementary analysis (relative to anhydrous substance):

Cld: C 52.40 H 5.50 N 8.91 Gd 14.29

Fnd: C 52.48 H 5.70 N 8.69 Gd 14.04

### EXAMPLE 28

a) 2-Dimethylamino-6-[(4-carboxy)benzoyl]-naphthalene

7.66 g (25 mmol) of the carboxylic acid that is described in Example 23b is dissolved in THF, and after 5.1 g (100 mmol) of lithium-dimethylamide (Aldrich) is added, it is refluxed for 8 hours and then stirred overnight at room temperature. Then, it is evaporated to the dry state, the residue is dispersed between ethyl acetate and aqueous citric acid solution, and the organic phase is finally washed with water and dried on sodium sulfate. The crude product is chromatographed on silica gel (mobile solvent:diisopropyl ether/glacial acetic acid=19:1).

Yield: 3.83 g (48% of theory)

Elementary analysis (relative to anhydrous substance):

Cld: C 75.22 H 5.37 N 4.39

Fnd: C 75.10 H 5.13 N 4.22

b) N_{α}[4-(2-dimethylamino-naphth-6-oyl)]benzoyl)-N_{ξ}(benzyloxycarbonyl)-lysine Methylester

1.60 g (5 mmol) of the carboxylic acid that is described in Example 28a above is dissolved in THF, and after 2 ml of triethylamine in acetone/ice bath is added, it is cooled to −15° C. At this temperature, 0.64 ml (5 mmol) of isobutyl chloroformate is added in drops, and it is stirred for 15 minutes. 1.62 g (5.5 mmol) of H-Lys(Z)-OMe (Slotin, L. A. et al., Can. J. Chem. 55, 4257-66 (1977)) in the least possible amount of THF is added in drops to this suspension at −15° C. It is allowed to stir for 2 more hours at this temperature and overnight at room temperature. Then, the suspension is evaporated to the dry state, and the residue is dispersed between dichloromethane and water. The organic phase is washed in succession with dilute hydrochloric acid and with sodium carbonate solution, and it is dried on sodium sulfate. After concentration by evaporation in vacuo, the residue is chromatographed on silica gel (mobile solvent:ethyl acetate/ethanol=9: 1).

Yield: 2.44 g (82% of theory)

Elementary analysis:

Cld: C 70.57 H 6.26 N 7.05

Fnd: C 70.80 H 6.41 N 7.27

c) N_{ξ}-Lysinamide conjugate of the gadolinium complex of 10-[4-carboxy-1-methyl-2-oxo-3-azabutyl]-1,4,7,10-tetraazacyclododecane-1,4,7-triacetic acid] and N_{α}[4-(2-dimethylamino-naphth-6-oyl)]benzoyl)-lysine

2.98 g (5 mmol) of the protected amino acid that is described in Example 28b above is dissolved in 25 ml of HBr/glacial acetic acid, and it is stirred for 30 minutes at room temperature. After 200 ml of the residue is added, the suspension that is produced is stirred overnight, the precipitate is filtered off, washed with ether and dried in a vacuum. The hydrobromide is then dissolved in methanol, mixed with 25 ml of 2N sodium hydroxide i solution and stirred overnight at room temperature. Then, the organic solvent is evaporated, the aqueous solution is set at pH 8.5 with dilute hydrochloric acid and evaporated to the dry state. The N_{α}[4-(2-dimethylamino-naphth-6-oyl)]benzoyl)-lysine that is produced is reacted without further purification (2.2 g, quantitative). 3 ml of triethylamine and then 2.2 g (5 mmol) of the described lysine derivative are added to 5.63 g (7.5 mmol) of the Gd complex active ester, described in Example 21d, in 75 ml of DMF. The suspension is stirred overnight at room temperature and then evaporated to the dry state. The residue is dissolved in water and absorptively precipitated for one hour with activated carbon. Carbon is filtered out, and it is ultrafiltered with an Amicon() ultrafiltration membrane YC 05 (cut-off: 500 Da). The residue is chromatographed on silica gel (mobile solvent:methanol/water=2:1). The combined fractions are concentrated by evaporation and taken up in a little water. The solution is mixed with 4 ml of anion exchanger IRA 410 (OH⁻-form), filtered, and the filtrate is freeze-dried.

Yield: 4.66 g (80% of theory)

Water content (Karl.Fischer): 9.0% Elementary analysis (relative to anhydrous substance):

Cld: C 51.03 H 5.42 N 10.58 Gd 14.85

Fnd: C 50.76 H 5.21 N 10.67 Gd 14.40

### EXAMPLE 29

a) 1-Methoxy-4-[(4-methoxycarbonyl)cyclohexylcarbonyl]-naphthalene

15.72 g (0.1 mol) of 1-methoxynaphthalene is dissolved under nitrogen in 130 ml of nitrobenzene, cooled to 12° C., and mixed with 17.3 g (0.13 mol) of aluminum chloride. Then, 26.6 g (0.13 mol) of 4-chlorocarbonyl-cyclohexanecarboxylic acid-methyl ester (Calaminus, W. et al., Z. Naturforsch. Volume 41, 1011-1014 (1986)), dissolved in nitrobenzene, is slowly added in drops at 12° C., stirred for 2 hours at this temperature and overnight at room temperature. The mixture is added to ice-cold concentrated hydrochloric acid and extracted several times with dichloromethane. The combined organic phase is dried on sodium sulfate and evaporated to the dry state in a vacuum. The residue is chromatographed on silica gel (mobile solvent:hexane/ethyl acetate=3:1).

Yield: 22.5 g (69% of theory) of a colorless solid

Elementary analysis:

Cld: C 73.60 H 6.79

Fnd: C 73.73 H 6.54

b) 1-Methoxy-4-[(4-carboxy)cyclohexylcarbonyl]-naphthalene

20.4 g (62.5 mmol) of the methyl ester that is described in Example 29a above is dissolved in dioxane, and after 250 ml of 2N NaOH is added, it is stirred overnight at room temperature. Then, the solution is evaporated to the dry state, and the residue is dispersed between ethyl acetate and dilute hydrochloric acid. The aqueous phase is extracted several times with ethyl acetate, and the combined organic phase is dried on sodium sulfate.

Yield: 20.3 g (quantitative)

Elementary analysis:

Cld: C 73.06 H 6.45

Fnd: C 72.81 H 6.22

c) 1-Methoxy-4-[(4-[2-benzyloxycarbonylaminoethyl]-carbamoyl)cyclohexylcarbonyl]-naphthalene

15.62 g (50 mmol) of the carboxylic acid that is described in Example 29b above is dissolved in THF, and after 20 ml of triethylamine in acetone/ice bath is added, it is cooled to −15° C. At this temperature, 6.43 ml (50 mmol) of isobutyl chloroformate is added in drops, and it is stirred for 15 minutes. In another vessel, 15.0 g (65 mmol) of N-(benzyloxycarbonyl) ethylenediamine hydrochloride (Eisenbrand, G. et al., Synthesis 1996, 1246-1258) in dichloromethane is taken up at the same time, shaken out twice with dilute sodium hydroxide solution, the organic phase is dried on sodium sulfate and evaporated to the dry state. The remaining oil is dissolved in THF and added in drops to the above-described reaction at −15° C. It is allowed to stir for 2 more hours at this temperature and overnight at room temperature. Then, the suspension is evaporated to the dry state, and the residue is dispersed between dichloromethane and water. The organic phase is washed in succession with dilute hydrochloric acid and with sodium carbonate solution and dried on sodium sulfate. After concentration by evaporation in vacuo, it is recrystallized from hexane.

Yield: 19.6 g (80% of theory) of a colorless solid

Elementary analysis:

Cld: C 71.29 H 6.60 N 5.73

Fnd: C 71.40 H 6.42 N 5.49

d) para-Nitrophenyl-active ester of the gadolinium complex of 10-[4-carboxy-2-oxo-3-azabutyl]-1,4,7,10-tetraazacyclododecane-1,4,7-triacetic Acid

9.79 g (15.9 mmol) of the gadolinium complex of 10-[4-carboxy-2-oxo-3-azabutyl]-1,4,7,10-tetraazacyclododecane-1,4,7-triacetic acid (DE 196 52 386, Schering A G, (priority: Dec. 4, 1996)) is dissolved in 70 ml of water, mixed with 1.28 ml (15.9 mmol) of pyridine, and the solution is freeze-dried. The lyophilizate that is obtained is taken up in 120 ml of pyridine, mixed with 7.24 g (23.8 mmol) of bis-(4-nitrophenyl)carbonate, and the suspension is stirred for 3 days at room temperature. Then, the solid is suctioned off, washed with pyridine and dichloromethane and dried at 40° C. in a vacuum.

Yield: 11.0 g (93.9% of theory)

Elementary analysis:

Cld: C 39.12 H 4.24 N 11.41 Gd 21.34

Fnd: C 39.61 H 4.30 N 11.77 Gd 20.87

e) Amide conjugate of the gadolinium complex of 10-[4-carboxy-2-oxo-3-azabutyl]-1,4,7,10-tetraazacyclododecane-1,4,7-triacetic acid] and 1-methoxy-4-[(4-[2-aminoethyl]-carbamoyl)cyclohexylcarbonyl]-naphthalene

2.44 g (5 mmol) of the protected amine that is described in Example 29c is dissolved in 25 ml of HMr/glacial acetic acid, and it is stirred for 30 minutes at room temperature. After 200 ml of ether is added, the suspension that is produced is stirred overnight, the precipitate is filtered off, washed with ether, and then dried in a high vacuum. The hygroscopic hydrobromide is further reacted without additional purification (2.18 g, quantitative).

3 ml of triethylamine and then 2.18 g (5 mmol) of the amine-hydrobromide are added to 5.53 g (7.5 mmol) of the Gd-complex-active ester in 75 ml of DMF that is described in Example 30d above. The suspension is stirred overnight at room temperature, and then it is evaporated to the dry state. The residue is dissolved in water and absorptively precipitated for one hour with activated carbon. Carbon is filtered out, and it is ultrafiltered to remove low-molecular components with an Amicon® ultrafiltration membrane YC 05 (cut-off: 500 Da). The residue is chromatographed on silica gel (mobile solvent:methanol/water=2:1). The combined fractions are concentrated by evaporation, taken up in water and freeze-dried.

Yield: 3.58 g (70% of theory) of a colorless powder.

Water content (Karl.Fischer): 7.0%

Elementary analysis (relative to anhydrous substance):

Cld: C 49.20 H 5.50 N 10.30 Gd 16.52

Fnd: C 49.00 H 5.41 N 10.53 Gd 16.22

### EXAMPLE 30

a) 1-Dimethylamino-4-[(4-carboxy)cyclohexylcarbonyl]-naphthalene

7.81 g (25 mmol) of the carboxylic acid that is described in Example 30b is dissolved in THF, and after 5.1 g (100 mmol) of lithium-dimethylamide (Aldrich) is added, it is refluxed for 8 hours and then stirred overnight at room temperature. Then, it is evaporated to the dry state, the residue is dispersed between ethyl acetate and aqueous citric acid solution, and the organic phase is finally washed with water and dried on sodium sulfate. The crude product is chromatographed on silica gel (mobile solvent:diisopropyl ether/glacial acetic acid=19:1).

Yield: 4.0 g (49% of theory)

Elementary analysis (relative to anhydrous substance):

Cld: C 73.82 H 7.12 N 4.30

Fnd: C 74.07 H 7.11 N 4.44

b) N_{α}[4-(1-Dimethylamino-naphth-4-oyl)]cyclohexylcarbonyl)-N_{ξ}(benzyloxycarbonyl)-lysine Methylester

1.63 g (5 mmol) of the carboxylic acid that is described in Example 30a above is dissolved in THF, and after 2 ml of triethylamine in acetone/ice bath is added, it is cooled to −15° C. At this temperature, 0.64 ml (5 mmol) of isobutyl chloroformate is added in drops, and it is stirred for 15 minutes. 1.62 g (5.5 mmol) of H-Lys(Z)-OMe (Slotin, L. A. et al., Can. J. Chem. 55, 4257-66 (1977)) in the least possible amount of THF is added in drops to this suspension at −15° C. It is allowed to stir for 2 more hours at this temperature and overnight at room temperature. Then, the suspension is evaporated to the dry state, and the residue is dispersed between dichloromethane and water. The organic phase is washed in succession with dilute hydrochloric acid and with sodium carbonate solution and dried on sodium sulfate. After concentration by evaporation in vacuo, the residue is chromatographed on silica gel (mobile solvent:ethyl acetate/ethanol=9:1).

Yield: 2.71 g (90% of theory)

Elementary analysis:

Cld: C 69.86 H 7.20 N 6.98

Fnd: C 69.61 H 7.08 N 6.89

c) N-Lysinamide conjugate of the gadolinium complex of 10-[4-carboxy-2-oxo-3-azabutyl]-1,4,7,10-tetraazacyclododecane-1,4,7-triacetic acid] and N_{α}[4-(1-dimethylamino-naphth-4-oyl)]cyclohexylcarbonyl)-lysine

3.01 g (5 mmol) of the protected amino acid that is described in Example 30b above is dissolved in 25 ml of HBr/glacial acetic acid, and it is stirred for 30 minutes at room temperature. After 200 ml of ether is added, the suspension that is produced is stirred overnight, the precipitate is filtered off, washed with ether and dried in a vacuum. The hydrobromide is then dissolved in methanol, mixed with 25 ml of 2N sodium hydroxide solution and stirred overnight at room temperature. Then, the organic solvent is evaporated, the aqueous solution is set at pH 8.5 with dilute hydrochloric acid and evaporated to the dry state. The N_{α}[4-(1-dimethylamino-naphth-4-oyl)]cyclohexylcarbonyl)-lysine that is produced is reacted without further purification (2.3 g, quantitative).

3 ml of triethylamine and then 2.3 g (5 mmol) of the lysine derivative that is described are added to 5.53 g (7.5 mmol) of the Gd complex active ester, described in Example 29d, in 75 ml of DMF. The suspension is stirred overnight at room temperature and then evaporated to the dry state. The residue is dissolved in water and absorptively precipitated for one hour with activated carbon. Carbon is filtered out, and ultrafiltered with an Amicon® ultrafiltration membrane YC 05 (cut-off: 500 Da). The residue is chromatographed on silica gel (mobile solvent:methano/water=2:1). The combined fractions are concentrated by evaporation and taken up in a little water. The solution is mixed with 4 ml of anion exchanger IRA 410 (OH—form), filtered, and the filtrate is freeze-dried.

Yield: 4.3 g (74% of theory)

Water content (Karl.Fischer): 10.0%

Elementary analysis (relative to anhydrous substance):

Cld: C 50.27 H 5.85 N 10.66 Gd 14.96

Fnd: C 50.40 H 5.62 N 10.40 Gd 14.55

### EXAMPLE 31

a) 1-Methoxy-4-[(4-methoxycarbonyl)naphth-1-oyl]-naphthalene

1.57 g (10 mmol) of 1-methoxynaphthalene is dissolved under nitrogen in 15 ml of nitrobenzene and mixed with 2.88 g (13 mmol) of indium chloride. Then, 3.23 g (13 mmol) of 1,4-naphthalene-dicarboxylic acid-monochloride-monomethyl ester (Frischkorn, Hans et al., Ger. Offen. (1978), DE 2715567), dissolved in nitrobenzene, is slowly added in drops, and stirred for 4 hours at 50° C. and overnight at room temperature. The mixture is added to ice-cold concentrated hydrochloric acid and extracted several times with dichloromethane. The combined organic phase is dried on sodium sulfate and evaporated to the dry state in a vacuum. The residue is chromatographed on silica gel (mobile solvent:hexane/ethyl acetate=4:1).

Yield: 2.64 g (71% of theory) of a colorless solid

Elementary analysis:

Cld: C 77.40 H 5.41

Fnd: C 77.42 H 5.19

b) 1-Methoxy-4-[(4-carboxy)naphth-1-oyl]-naphthalene

2.33 g (6.25 mmol) of the methyl ester that is described in Example 3 la above is dissolved in dioxane, and after 25 ml of 2N NaOH is added, it is stirred overnight at room temperature. Then, the solution is evaporated to the dry state, and the residue is dispersed between ethyl acetate and dilute hydrochloric acid. The aqueous phase is extracted several times with ethyl acetate, and the combined organic phase is dried on sodium sulfate.

Yield: 2.2 g (quantitative)

Elementary analysis:

Cld: C 77.08 H 5.06

Fnd: C 76.81 H 5.16

c) 1-Methoxy-4-[(4-[2-benzyloxycarbonylaminoethyl]-carbamoyl)naphth-1-oyl]-naphthalene

1.79 g (5 mmol) of the carboxylic acid that is described in Example 3 lb above is dissolved in THF, and after 2 ml of triethylamine in acetone/ice bath is added, it is cooled to −15° C. At this temperature, 0.64 ml (5 mmol) of isobutyl chloroformate is added in drops, and it is stirred for 15 minutes. In another vessel, 1.5 g (6.5 mmol) of N-(benzyloxycarbonyl) ethylenediamine hydrochloride (Eisenbrand, G. et al., Synthesis 1996, 1246-1258) in dichloromethane is taken up at the same time, shaken out twice with dilute sodium hydroxide solution, the organic phase is dried on sodium sulfate and evaporated to the dry state. The remaining oil is dissolved in THF and added in drops to the above-described reaction at −15° C. It is allowed to stir for 2 more hours at this temperature and overnight at room temperature. Then, the suspension is evaporated to the dry state, and the residue is dispersed between dichloromethane and water. The organic phase is washed in succession with dilute hydrochloric acid and with sodium carbonate solution, and it is dried on sodium sulfate. After concentration by evaporation in vacuo, it is recrystallized from hexane.

Yield: 2.25 g (84% of theory) of a colorless solid

Elementary analysis:

Cld: C 73.86 H 6.01 N 5.22

Fnd: C 73.53 H 5.81 N 5.23

d) Amide Conjugate of the Gadolinium Complex of 10-[4-carboxy-1-methyl-2-oxo-3-azabutyl]-1,4,7,10-tetraazacyclododecane-1,4,7-triacetic acid] and 1-methoxy-4-[(4-[2-aminoethyl]-carbamoyl)naphth-1-oyl]-naphthalene

2.68 g (5 mmol) of the protected amine that is described in Example 3 ic above is dissolved in 3 ml of HBr/glacial acetic acid, and it is stirred for 30 minutes at room temperature. After 50 ml of ether is added, the suspension that is produced is stirred overnight, the precipitate is filtered off, washed with ether and then dried in a high vacuum. The hygroscopic hydrobromide is further reacted without additional purification (2.42 g, quantitative). 3 ml of triethylamine and then 2.42 g (5 mmol) of the amine-hydrobromide are added to 5.63 g (7.5 mmol) of the Gd-complex active ester, described in Example 21d, in 75 ml of DMF. The suspension is stirred overnight at room temperature and then evaporated to the dry state. The residue is dissolved in water and absorptively precipitated for one hour with activated carbon. Carbon is filtered out and ultrafiltered to remove low-molecular components with an Amicon() ultrafiltration membrane YC 05 (cut-off: 500 Da). The residue is chromatographed on silica gel (mobile solvent:methanol/water=3:1). The combined fractions are concentrated by evaporation, taken up in water and freeze-dried.

Yield: 4.31 g (78% of theory) of a colorless powder.

Water content (Karl.Fischer): 8.3%

Elementary analysis (relative to anhydrous substance):

Cld: C 52.21 H 5.18 N 9.69 Gd 15.54

Fnd: C 52.05 H 4.94 N 9.88 Gd 15.12

### EXAMPLE 32

a) N_{α}[4-(1-Methoxynaphth-4-oyl)naphth-1-oyl]-N_{ξ}(t-butoxycarbonyl)-lysine t-butylester

17.92 g (50 mmol) of the carboxylic acid that is described in Example 31b is dissolved in THF, and after 20 ml of triethylamine in acetone/ice bath is added, it is cooled to −15° C. At this temperature, 6.43 ml (50 mmol) of isobutyl chloroformate is added in drops, and it is stirred for 15 minutes. 16.63 g (55 mmol) of H-Lys(Boc)-OtBu (Wakimasu, M. et al., Chem. Pharm. Bull. 29, 2592-2597 (1981)) in the least possible amount of THF is added in drops to this suspension at −15° C. It is allowed to stir for 2 more hours at this temperature and overnight at room temperature. Then, the suspension is evaporated to the dry state, and the residue is dispersed between dichloromethane and water. The organic phase is washed in succession with dilute hydrochloric acid and with sodium carbonate solution, and it is dried on sodium sulfate. After concentration by evaporation in vacuo, the residue is chromatographed on silica gel (mobile solvent:ethyl acetate/ethanol 10:1).

Yield: 34.4 g (85% of theory) of a colorless solid

Elementary analysis:

Cld: C 71.45 H 6.63 N 4.39

Fnd: C 71.39 H 6.41 N 4.28

b) N_{α}[4-(1-Methoxynaphth-4-oyl)naphth-1-oyl]-lysine

3.19 g (5 mmol) of the protected amino acid that is described in Example 32a above is suspended in 30 ml of trifluoroacetic acid, and it is stirred for 2 hours at room temperature. After 200 ml of ether is added, the suspension is stirred overnight, the precipitate is filtered off, washed carefuilly with ether and then dried in a high vacuum to a constant weight in the presence of KOH.

Yield: 2.5 g (quantitative) of a colorless solid

Elementary analysis:

Cld: C 71.29 H 6.60 N 5.73

Fnd: C 71.04 H 6.41 N 5.70

c) N,-Lysinamide conjugate of the gadolinium complex of 10-[4-carboxy-1-methyl-2-oxo-3-azabutyl]-1,4,7,10-tetraazacyclododecane-1,4,7-triacetic acid] and N_{α}[4-(1-methoxynaphth-4-oyl)naphth-1-oyl]-lysine

3 ml of triethylamine and then 2.5 g (5 mmol) of the lysine derivative that is described in Example 32b above are added to 5.63 g (7.5 mmol) of the Gd complex active ester, described in Example 21 d, in 75 ml of DMF. The suspension is stirred overnight at room temperature and then evaporated to the dry state. The residue is dissolved in water and absorptively precipitated for one hour with activated carbon. Carbon is filtered out and ultrafiltered with an Amicon(ultrafiltration membrane YC 05 (cut-off: 500 Da). The residue is chromatographed on silica gel (mobile solvent:methanol/water=3:1). The combined fractions are concentrated by evaporation, taken up ,in water and freeze-dried.

Yield: 4.7 g (78% of theory) of a colorless powder.

Water content (Karl.Fischer): 8.5%

Elementary analysis (relative to anhydrous substance):

Cld: C 52.40 H 5.50 N 8.91 Gd 14.29

Fnd: C 52.18 H 5.35 N 8.88 Gd 13.96

### EXAMPLE 33

a) 2-Bromo-terephthalic acid-mono-tbutyl Monoester

50 g (184.4 mmol) of 2-bromo-4-methyl-benzoic acid-tbutyl ester [produced from the acid chloride by esterification with tbutanol analogously to Org. Synth. Coll. Vol. II, 142 (1955); IV., 263 (1963)] and 63.2 g (400 mmol) of potassium permanganate are suspended in 400 ml of water and heated to 50° C. It is stirred for 8 hours at 50° C. Precipitated manganese dioxide is filtered out, and the pH of the filtrate is set at 2.8 with 2N sulfuric acid. It is extracted twice with 300 ml of ethyl acetate, the organic phases are combined, dried on magnesium sulfate and evaporated to the dry state in a vacuum.

Yield: 54.2 g (98% of theory) of a colorless solid

Elementary analysis:

Cld: C 48.02 H 4.03 Br 26.62

Fnd: C 48.14 H 4.10 Br 26.81

b) 2-Bromo-terephthalic acid-4-(8-quinolinylester)-tbutylester

50 g (166.6 mmol) of the title compound of Example 33a and 24.2 g (166.6 mmol) of 8-hydroxyquinoline are dissolved in 500 ml of dichloromethane. At 0° C., 34.4 g (166.6 mmol) of N,N′-dicyclohexylcarbodiimide is added, and it is stirred for 5 hours at 0° C. It is allowed to come to room temperature, the precipitated urea is filtered off, and the filtrate is evaporated to the dry state in a vacuum. The residue is recrystallized from diethyl ether/n-hexane.

Yield: 60.0 g (87% of theory)

Elementary analysis:

Cld: C 60.88 H 4.38 Br 19.29

Fnd: C 60.99 H 4.50 Br 19.16

c) 2-Methoxy-6-[(3-bromo-4-tbutyloxycarbonyl)-benzoyl]-naphthalene

A solution of 62.14 g (150 mmol) of the title compound of Example 33b, dissolved in 200 ml of tetrahydrofuran, is added in drops at −50° C. to a Grignard solution, produced from 3.65 g (150 mmol) of magnesium and 35.69 g (150 mmol) of 2-methoxy-6-bromo-naphthalene in 200 ml of tetrahydrofuran. It is stirred for 3 hours at −50° C. and then allowed to come to room temperature. 800 ml of water is added, and it is set at pH 3.5 by adding citric acid. It is extracted twice with 500 ml of diethyl ether. The combined organic phases are dried on magnesium sulfate and evaporated to the dry state in a vacuum. The residue is chromatographed on silica gel (mobile solvent:hexane/ethyl acetate=20:1).

Yield: 35.8 g (81% of theory) of a colorless solid

Elementary analysis:

Cld: C 62.60 H 4.80 Br 18.11

Fnd: C 62.48 H 4.91 Br 18.02

d) 2-Methoxy-6-{[2-methoxycarbonyl)-ethyl-4-tbutyloxycarbonyl]-benzoyl} -naphthalene

25.8 g (300 mmol) of acrylic acid methyl ester is added to a mixture that consists of 44.13 g (100 mmol) of the title compound of Example 33c, 11.56 g (10 mmol) of tetrakis(triphenyl-phosphine)-palladium (Tetrahedron Lett. 1992, 33, 4859; J. Am. Chem. Soc., 1992, 114, 7292) and 30.4 g (300 mmol) of triethylamine in 400 ml of dimethylformamide, and it is stirred for 5 hours at 70° C. 500 ml of water is added, and it is allowed to come to room temperature. It is set at a pH of 6 by adding 1N hydrochloric acid and extracted twice with 500 ml of diethyl ether. The combined organic phases are evaporated to the dry state in a vacuum, the residue is dissolved in 300 ml of methanol, and 5 g of palladium catalyst (10 Pd/C) is added. It is hydrogenated at room temperature under normal pressure. Catalyst is filtered out, and the filtrate is evaporated to the dry state in a vacuum. The residue is chromatographed on silica gel (mobile solvent:hexane/ethyl acetate=20:1).

Yield: 33.64 g (75% of theory) of a colorless solid

Elementary analysis:

Cld: C 72.30 H 6.29

Fnd: C 72.42 H 6.40

e) 2-Methoxy-6-{[3-(2-hydrazinocarbonyl)-ethyl-4-tbutyloxycarbonyl]-benzoyl}-naphthalene

5.51 g (110 mmol) of hydrazine hydrate is added to a solution that consists of 24.43 g (50 mmol) of the title compound of Example 33d, dissolved in 100 ml of methanol, and it is refluxed for 8 hours. Half of the solvent is distilled off in a vacuum and cooled to 0° C. In this case, the title compound crystallizes out. It is filtered off and dried in a vacuum at 40° C.

Yield: 40.8 g (91% of theory) of a colorless solid

Elementary analysis:

Cld: C 69.63 H 6.29 N 6.25

Fnd: C 69.76 H 6.38 N 6.37

f) Hydrazide Conjugate from the Gadolinium Complex of 10-[4-carboxy-1-methyl-2-oxo-3-azabutyl]-1,4,7,10-tetraazacyclododecane-1,4,7-triacetic acid] and 2-methoxy-6-{[3-(2-hydrazinocarbonyl)-ethyl-4-tbutyloxycarbonyl]-benzoyl}-naphthalene

20 g (31.76 mmol) of the Gd complex of 10-(4-carboxy-1-methyl-2-oxo-3-aza-butyl)-1,4,7,10-tetraazacyclododecane-1,4,7-triacetic acid (Gd-Gly-Me-DOTA, DE 19652386), 2.69 g (63.52 mmol) of lithium chloride and 3.66 g (31.76 mmol) of N-hydroxysuccinimide are dissolved in 200 ml of dimethyl sulfoxide while being heated slightly. Then, 14.24 g (31.76 mmol) of the title compound of Example 33e is added, and it is stirred for 20 minutes at room temperature. It is cooled to 10° C., 9.83 g (47.64 mmol) of N,N′-dicyclohexylcarbodiimide is added, and it is stirred overnight at room temperature. The reaction solution is poured into a mixture that consists of 1000 ml of acetone/1000 ml of diethyl ether, and the precipitated solid is filtered off. Further purification is carried out by chromatography on silica gel RP-18 (mobile solvent: gradient that consists of water/acetonitrile/tetrahydrofuran. After the fractions are concentrated by evaporation, 27.95 g (83% of theory) of a colorless, amorphous solid is obtained.

Water content: 8.5%

Elementary analysis (relative to anhydrous substance):

Cld: C 50.98 H 5.32 Gd 14.83 N 9.25

Fnd: C 51.11 H 5.44 Gd 14.95 N 9.37

g) Hydrazide Conjugate that Consists of the Gadolinium Complex of 10-[4-carboxy-1-methyl-2-oxo-3-azabutyl]-1,4,7,10-tetraazacyclododecane-1,4,7-triacetic acid] and 2-methoxy-6-{[3-(2-hydrazinocarbonyl)-ethyl-4-carboxy]-benzoyl)-naphthalene, sodium salt

21.21 g (20 mmol) of the title compound of Example 33fis dissolved in 150 ml of trifluoroacetic acid, and it is stirred for 2 hours at room temperature. It is evaporated to the dry state in a vacuum, and the residue is purified by chromatography on silica gel RP-18 (mobile solvent: gradient that consists of water/acetonitrile/tetrahydrofuran). After the fractions are concentrated by evaporation in a vacuum, the residue is dissolved in 400 ml of water, and the pH is set at 7.6 by adding 2N sodium hydroxide solution. The solution is filtered and freeze-dried.

Yield: 19.46 g (95% of theory) of a colorless, amorphous powder

Elementary analysis (relative to anhydrous substance):

Cld: C 48.09 H 4.43 N 9.57 Gd 15.36 Na 2.24

Fnd: C 48.00 H 4.50 N 9.69 Gd 15.51 Na 2.31

### EXAMPLE 34

a) Hydrazide Conjugate from the Gadolinium Complex of 10-[4-carboxy-1-methyl-2-oxo-3-azabutyl]-1,4,7,10-tetraazacyclododecane-1,4,7-triacetic acid] and 2-methoxy-6-{ [3-(2-hydrazinocarbonyl)-ethyl-4-carboxy]-cyclohexylcarbonyl}-naphthalene, Sodium Salt

10 g of Raney nickel is added to a solution that consists of 21.21 g (20 mmol) of the title compound of Example 33fin 200 ml of water, and it is hydrogenated at 50° C. and a pressure of 3 bar of hydrogen in an autoclave. Nickel is filtered out, and the filtrate is evaporated to the dry state. The residue is chromatographed on silica gel RP-18 (mobile solvent: gradient that consists of water/acetonitrile). The fractions are concentrated by evaporation in a vacuum.

Yield: 18.95 g (92% of theory) of a colorless, amorphous solid

Elementary analysis (relative to anhydrous substance):

Cld: C 47.80 H 4.99 N 9.52 Gd 15.27 Na 2.23

Fnd: C 47.65 H 5.10 N 9.66 Gd 15.40 Na 2.30

### EXAMPLE 35

Production of Manganese(II) and Iron(III) Complexes

The production of manganese and iron complexes is carried out from the above-described gadolinium complexes. In this connection, e.g., 10 mmol of a Gd-complex compound in 100 ml of water is dissolved and 2 equivalents of oxalic acid are added per Gd-ion. It is mixed with 1 ml of concentrated hydrochloric acid and stirred for 3 hours at 70° C.

It is cooled to 0° C., precipitated gadolinium oxalate is filtered out, and the filtrate is evaporated to the dry state in a vacuum. The residue is purified on RP-18 (mobile solvent: gradient that consists of water/acetonitrile/tetrahydrofuran).

The complexing agents that are thus obtained are dissovled in water and reacted either with freshly precipitated iron(III) hydroxide or with manganese carbonate at 80° C. (for 3 hours). In the case of manganese, the pH of the solution is ultimately brought to 7.4 (with NaOH).

The solutions are filtered and then freeze-dried.

The iron(III) complexes are obtained as dark-yellow to brown amorphous solids. The manganese (II) complexes are colorless, amorphous solids.

### EXAMPLE 36

Determination of Relaxivity R1 [Lmmol⁻¹ s⁻¹] and R2 [Lmmol⁻¹ s⁻¹]

Device Minispec PC 20

Measurement at 40° C.; 0.47 tesla

T1-sequence 180°-TI-90°, inversion recovery

| | Concentration in the | | | |
|---|---|---|---|---|
| Substance | feedstock [mmol/L] | Medium | R1 | R2 |
| 4 | 0.22-0.88 | bovine | 17.0 ± 0.1 | 20.2 ± 0.6 |
| | n = 3 | plasma | | |
| 11 | 0.22-0.88 | bovine | 22.8 ± 0.8 | 29.2 ± 1.1 |
| | n = 3 | plasma | | |
| 1c, DE | 0.05-0.5 | bovine | 15.3 ± 0.3 | 16.3 ± 0.2 |
| 4232925 | n = 3 | plasma | | |

### EXAMPLE 37

MRI Experiments on Animals with Induced Myocardial Infarction

The build-up in the myocardial infarction and the necrosis-selective enhancement were studied after one-time intravenous administration of the substance of example 13 to animals with experimentally produced myocardial infarction.

The induction of the myocardial infarction was carried out on anesthetized (Domitor®/Dormicum®, i.m.) rats (Shoe. Wistar, Schering SPF, about 300 g of body weight) by occlusion of the left coronary artery. The contrast medium administration (dose: 100 [mol of Gd per kg of body weight) was carried out in each case about 24 hours after the induction of infarction. The animals were sacrificed about 24 hours after substance administration (in the MR tomograph) by an overdose of narcotics, and studied immediately using M tomography (Siemens Allegra, 1.5 tesla; SE sequence, T_{R}: 400 ms, T_{E}: 6 ms, NEX: 4, Ma: 128*128, FOV: 7*7 cm, SD≈2.5 mm, 1 layer each axially). To verify the infarction (size and position), the heart was prepared immediately after the MRT experiments, sliced into disks and then NBT (nitro blue tetrazolium chloride) vital staining was performed. For the quantification of the substance build-up, the vital (stained) myocardial areas were separated from the necrotic (unstained) areas based on the staining reaction, and they were correspondingly worked up to determine the metal content. The determination of the metal content was carried out using “inductively coupled plasma atomic emission spectroscopy” (ICP-AES). The infarction build-up was calculated from the Gd concentrations in the tissues as follows: infarction build-up =Gd concentration in the infarction area/Gd concentration in the “normal” myocardium.

Without substance administration, the infarcted area in the MR tomogram cannot be distinguished from the “normal” myocardium, since both areas are presented isointensively

[image]

[image]

1. Compounds of general formula I
Ar-(L-K)ₙ (I)
in which
K means a cyclic non-radioractive metal complex of the DOTA type,
L means a linker,
Ar means an aromatic radical, which contains a polycondensated aromatic hydrocarbon, and
n means the numbers 1 or 2.

2. Compounds according to claim 1, characterized in that Ar stands for a radical 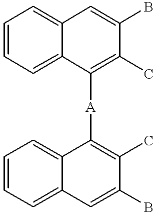
with the meaning
A: a direct bond,
a methylene group —CH₂—,
a dimethylene ether group —CH₂—O—CH₂—,
B: a hydrogen atom,
a carbonyl group —CO—,
C: a hydroxyl group —OH,
an oxygen group —O—.
an ether group —OR¹, in which R¹ means an alkyl radical with 1-3 carbon atoms, whereby substituents B and C in the molecule are respectively identical,
for a radical 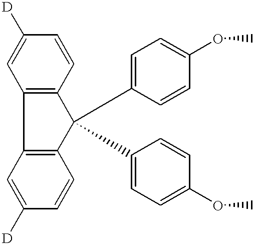
with the meaning
D: a hydrogen atom,
an ether group —OR, with R¹ in the above mentioned meaning,
for radical 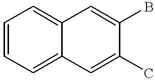
with the meaning
B and C as described above,
for a radical 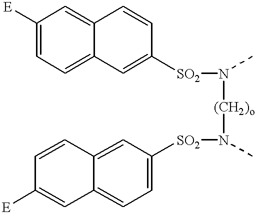
with the meaning
E: a hydrogen atom,
An ether group —OR¹,
A dialkylamino group N(R¹)₂, whereby R¹ has the above-mentioned meaning,
o: a number between 2-10,
for a radical 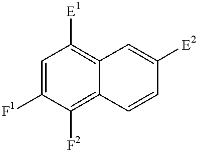
with the meaning
E¹, E²: independently of one another, in the meaning of E,
F¹, F²: independently of one another, for a hydrogen atom H or the radicals 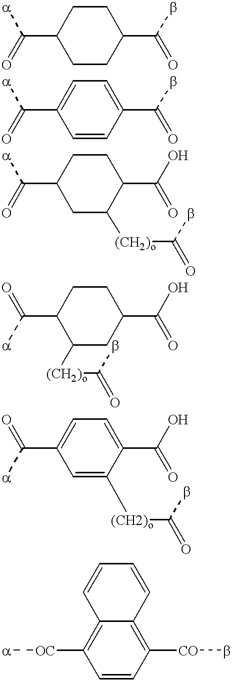
with o in the above-mentioned meaning,
and provided that one of substituents F¹ or F² stands for a hydrogen atom and that a refers to the binding site orientated to the aromatic compounds and β refers to the binding site orientated to the metal complex.

3. Compounds according to claim 1, wherein
L stands for a linker in the meaning of a hydrazine group —NHNH, a C₂-C₂₀ carbon chain with terminal —NH, which can be linear or branched, saturated or unsaturated and optionally is interrupted by 1-6 oxygen atoms, 1-2 phenylene groups, 1-2 cyclohexylidene groups, 1-2 groups —NH—CO— or —CONH—, 1-2 groups —CH₂CONHNH— or —NHNHCOCH₂— and optionally is substituted with 1-2 hydroxyl groups, with 1-2 methoxy groups, with 1-2 carboxy groups.

4. Compounds according to claim 1, wherein
K stands for a metal complex of general formula II 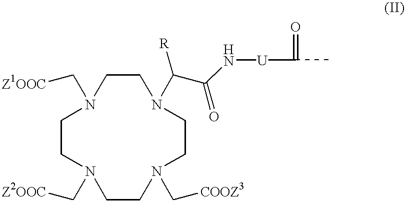
with the meaning
R: a hydrogen atom,
a methyl group,
Z¹, Z², Z³: a metal ion equivalent of the atomic numbers 25, 26 as well as 58-70,
U: a C₁-C₁₀ carbon chain, linear or branched, saturated or unsaturated, optionally interrupted by 1-2 oxygen atoms, by a phenylene group, by a cyclohexylidene group, by one or two groups —NH—CO— or —CONH—, optionally substituted with one to two —CO₂H groups, with one to three hydroxyl groups, one to three methoxy or alkoxy groups,
or for a metal complex of general formula III 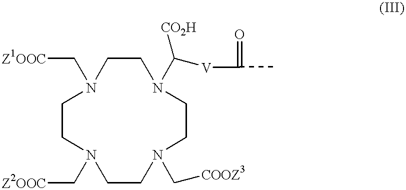
with the meaning
Z¹, Z², Z³: as indicated above.
V: a phenylene, phenylenoxymethyl- -δ-C₆H₄—O—CH₂-γ group whereby γ indicates the binding site orientated to the aromatic compound and δ indicates the binding site orientated to the metal complex,
a C₁-C₂₀ carbon chain, linear or branched, saturated or unsaturated, optionally interrupted by one to two oxygen atoms, by a phenylene group, by a cyclohexylidene group, by one or two groups —NH—CO— or CONH—, optionally substituted with one to two —CO₂H groups with one to three hydroxyl groups, one to three methoxy or alkoxy groups.

5. Compounds according to claim 1, wherein
L stands for a radical
—NH—NH—
γ-CH₂—CONH—NHδ
—NH—CH₂CH₂—NH—
—NH CH₂CH₂CH₂CH₂—NH—
—NH—(CH₂)₃—NH—
—NH—(CH₂)₅—NH—
—NH—(CH₂)₂—O—(CH₂)₂—NH—
γ-NH—(CH₂)k-CONH—(CH₂)m-NH-δ mit k=1-10; m=0-10,
γ-NH—(CH₂CH₂O)₂—CH₂CH₂NH-δwith
γ-NH—CH—(CH₂)₄—NH-δ
COOH
—NH—CH₂CHOH—CH₂NH— 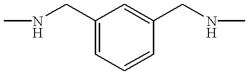
γ-NH—(CH₂CH₂O)₃CH₂—NH-δ, 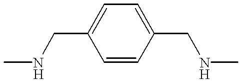 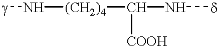
and γ ans δ have the meanings given in claim 4.

6. Compounds according to claims 1 and 4, wherein
U stands for a group
—CH₂—
—CH₂CH₂—
—C₆H₄—
—CH₂—O—CH₂CH₂—.

7. Compounds according to claim 1, wherein
V stands for a group
—CH₂—O—C₆H₄—
—C₆H₄—
—CH₂CH₂—
—CH₂—.

8. Compounds according to claim 1, wherein the central ion of metal complex K is a gadolinium ion, iron ion or manganese ion.

9. Pharmaceutical agent that contains at least one compound according to claim 1, optionally with the additives that are commonly used in galenicals.

10. Use of at least one compound according to claim 1 for the production of agents for MR imaging of necrosis and infarction.

11. Process for the production of compounds according to claim 1, wherein compounds of general formula IV
Ar(L-H)ₙ (IV)
are reacted with complexes or complexing agents of general formula V
K-X′ (V)
in which
Ar, L, K and n have the meaning that is mentioned in claim 1, and X′ stands for a hydroxy group or a group that activates the carboxylic acid, and optionally then is reacted (if K-X′ stands for a complexing agent) in a way that is known in the art with a metal oxide or metal salt of an element of atomic numbers 25, 26 or 58-70 and optionally then acid hydrogen atoms that are still present in the complexes that are thus obtained are substituted completely or partially by cations of inorganic and/or organic bases, amino acids or amino acid amides.

12. Process for the production of the pharmaceutical agents according to claim 9, wherein the metal complex that is dissolved or suspended in water or physiological salt solution, optionally with the additives that are commonly used in galenicals, is brought into a form that is suitable for enteral or parenteral administration.
